(19) United States
(12) Patent Application Publication
(10) Pub. No.: US 2026/0060645 A1
(43) Pub. Date: Mar. 5, 2026

(54) METHODS AND SYSTEMS FOR TRACKING TISSUE MOTION USING COVARIANCE AND ANTIALIAS PARAMETRIC TOOLS

(71) Applicant: OtoNexus Medical Technologies, Inc., Bellevue, WA (US)

(72) Inventors: Mark A. MOEHRING, Bellevue, WA (US); Andrew S. KESSLER, Bellevue, WA (US); Charles M. BOHLIN, Bellevue, WA (US)

(73) Assignee: OtoNexus Medical Technologies, Inc., Bellevue, WA (US)

(21) Appl. No.: 19/380,285

(22) Filed: Nov. 5, 2025

Related U.S. Application Data
(63) Continuation of application No. PCT/US2024/028427, filed on May 8, 2024.
(60) Provisional application No. 63/500,833, filed on May 8, 2023.

(51) Int. Cl. A61B 8/12; A61B 8/00

(52) U.S. Cl. CPC A61B 8/12; A61B 8/5215; A61B 8/5276

(57) ABSTRACT

A method of determining a time-dependent position of a tissue in response to a stimulus may include receiving ultrasound data derived from an ultrasound waveform reflected from the tissue; calculating from the ultrasound data a covariance matrix from a group of depths and between a plurality pulse periods of the ultrasound data; and calculating one or more eigenvectors and associated eigenvalues of the covariance matrix, wherein the one or more eigenvectors represent a first trace of a position of the tissue.

### CROSS-REFERENCE

This application is a continuation of PCT Application No. PCT/US24/28427, filed May 8, 2024, which claims the benefit of U.S. Provisional Application No. 63/500,833, filed May 8, 2023, which is incorporated by reference herein in its entirety.

### BACKGROUND

Pneumatic otoscopy is a medical examination that allows determination of the mobility of a tympanic membrane of a patient in response to pressure changes (e.g., airflow). A healthy tympanic membrane moves in response to pressure. Immobility may be due to fluid in the middle ear. Otitis media with effusion is characterized by the presence of fluid adjacent the tympanic membrane. Accordingly, establishing a diagnosis of otitis media with effusion may be aided by pneumatic otoscopy.

### SUMMARY

Pneumatic otoscopy using ultrasound may present both advantages and challenges. For example, ultrasound typically requires a coupling medium: however, filing an ear canal with a coupling medium may not be viable. Air-coupled ultrasound, however, may be difficult. For example, air may typically have an acoustic impendence with significant mismatch to a transducer and/or a material to be measure. The above challenges may lead to less sensitive measurements. Further, ultrasound measurements may have characteristically low signal to noise during or near in time to the application of a pneumatic stimulus. Accordingly, determination of a position of a tympanic membrane during or near in time to the application of a pneumatic stimulus may be challenging.

Methods and systems disclosed herein address at least some of the above identified challenges. Autoregression and covariance analysis separately or in combination may be used to improve extraction of tissue motion and position in this region at least in part by exploiting the periodicity of the received ultrasound signal.

In an aspect, disclosed herein are methods of determining a time-dependent position of a tissue in response to a stimulus, the methods comprising: (a) receiving ultrasound data, wherein the ultrasound data is derived from an ultrasound waveform reflected from the tissue; (b) calculating from the ultrasound data a covariance matrix from a group of depths and for a plurality of pulse periods of the ultrasound data; (c) calculating one or more eigenvectors and associated eigenvalues of the covariance matrix; and (d) determining a first trace of a position of the tissue associated with a content of a principal eigenvector of the one or more eigenvectors.

In some embodiments, methods of determining a time-dependent position of a tissue in response to a stimulus further comprise analyzing a frequency content of the ultrasound data across the plurality of pulse periods. In some embodiments, methods of determining a time-dependent position of a tissue in response to a stimulus further comprise calculating a second trace of a position of the tissue based at least in part on the frequency content.

In some embodiments, methods of determining a time-dependent position of a tissue in response to a stimulus further comprise outputting an indication of a disease state of the tissue in response to the second trace of the position of the tissue. In some embodiments, methods of determining a time-dependent position of a tissue in response to a stimulus further comprise outputting an indication of a health state of the tissue in response to the second trace of the position of the tissue. In some embodiments, methods of determining a time-dependent position of a tissue in response to a stimulus further comprise outputting an indication of an undetermined state of the tissue in response to the second trace of the position of the tissue.

In some embodiments, the plurality of pulse periods is sequential. In some embodiments, the tissue is a tympanic membrane.

In some embodiments, methods of determining a time-dependent position of a tissue in response to a stimulus further comprise outputting an indication of a disease state in response to the first trace of the position of the tissue. In some embodiments, methods of determining a time-dependent position of a tissue in response to a stimulus further comprise outputting an indication of a health state in response to the first trace of the position of the tissue. In some embodiments, methods of determining a time-dependent position of a tissue in response to a stimulus further comprise outputting an indication of an undetermined state of the tissue in response to the first trace of the position of the tissue.

In some embodiments, each pulse period of the plurality of pulse periods is associated with a related covariance matrix from a plurality of related covariance matrices. In some embodiments methods of determining a time-dependent position of a tissue in response to a stimulus further comprise calculating a set of displacement vectors for the plurality of related covariance matrices. In some embodiments, the plurality of related covariance matrices is associated with pulse periods across contiguous adjacent depths of the ultrasound data. In some embodiments, the set of displacement vectors for the plurality of related covariance matrices are calculated based on a target quality signal ratio. In some embodiments, the target quality signal ratio is a value between 0.00 and 1.00. In some embodiments, methods of determining a time-dependent position of a tissue in response to a stimulus further comprise stitching together segments of the set of displacement vectors to generate a time trace the position of the tissue. In some embodiments, the time trace of the position of the tissue is the first trace.

In some embodiments, methods of determining a time-dependent position of a tissue in response to a stimulus further comprise analyzing one or more eigenvectors of the covariance matrix. In some embodiments, the one or more eigenvectors each comprise an orbital rotation associated with a phase of the ultrasound data. In some embodiments, methods of determining a time-dependent position of a tissue in response to a stimulus further comprise applying a bias to each of the one or more eigenvectors such that the eigenvector circles an origin in phase space. In some embodiments, methods of determining a time-dependent position of a tissue in response to a stimulus further comprise using a resulting biased eigenvector to calculate a second trace of the position of the tissues. In some embodiments, analyzing the one or more eigenvectors further comprises analyzing membrane motion information of the one or more eigenvectors. In some embodiments, analyzing the one or more eigenvectors further comprises repeating the analysis in 10 millisecond (ms) steps in an adjustable 20 ms analysis window. In some embodiments, analyzing the one or more eigenvectors further comprises analyzing depth range values. In some embodiments, analyzing the one or more eigenvectors further comprises creating a set of principal eigenvectors at each 10 millisecond (ms) step. In some embodiments, analyzing the one or more eigenvectors further comprises creating a set of principal eigenvectors within each 20 ms analysis window.

In some embodiments, the stimulus is a pneumatic excitation. In some embodiments, the pneumatic excitation is an air puff.

In some embodiments, methods of determining a time-dependent position of a tissue in response to a stimulus further comprise analyzing a frequency content of the ultrasound data across the plurality of pulse periods and determining one or more regions where the first trace comprises a non-physical tissue position or movement. In some embodiments, methods of determining a time-dependent position of a tissue in response to a stimulus further comprise analyzing a frequency content of the ultrasound data across the plurality of pulse periods and substituting at least a portion of the second trace for the first trace in the one or more regions. In some embodiments, determining one or more regions where the first trace comprises a non-physical tissue position or movement comprises utilizing autoregression. In some embodiments, the autoregression is a third order autoregression. In some embodiments, the autoregression is higher than a third order autoregression.

In another aspect, disclosed herein are methods of improving accuracy of a time-dependent position determination of a tissue in response to a stimulus, the methods comprising: applying autoregression to a signal trace to generate a plurality of poles; eliminating or selecting a pole of the plurality of poles using a feature of the plurality of poles; and generating an improved signal trace of a position of the tissue from remaining or selected poles of the plurality of poles.

In some embodiments, the autoregression is applied to one or more points of the signal trace. In some embodiments, the autoregression is a third order autoregression. In some embodiments, the autoregression is higher than a third order autoregression. In some embodiments, the order of the autoregression corresponds with the number of frequency peaks of the signal trace that are separated. In some embodiments, the signal trace comprises a covariance motion detection trace. In some embodiments, the autoregression is applied to one or more eigenvectors of the covariance motion detection trace. In some embodiments, the methods of improving accuracy of a time-dependent position determination of a tissue in response to a stimulus further comprise estimating a mean frequency of the signal trace using an angle value between each of the plurality of poles and a center of a complex unit circle of a complex plane. In some embodiments, the angle value is calculated by taking an inverse tangent over the imaginary component of the pole of the real component of the pole.

In some embodiments, the methods of improving accuracy of a time-dependent position determination of a tissue in response to a stimulus further comprise estimating variance of the frequency of the signal trace using a radius value between each of the plurality of poles and a center of a complex unit circle of a complex plane. In some embodiments, the methods of improving accuracy of a time-dependent position determination of a tissue in response to a stimulus further comprise estimating a velocity represented by the signal trace using a radius value between each of the plurality of poles and a center of a complex unit circle of a complex plane.

In some embodiments, the methods of improving accuracy of a time-dependent position determination of a tissue in response to a stimulus further comprise plotting a velocity trace over time of the signal trace by plotting the angle between the plurality of poles and a center of a complex unit circle of a complex plane.

In some embodiments, the methods of improving accuracy of a time-dependent position determination of a tissue in response to a stimulus further comprise calculating one or more displacement values between a position of the velocity trace and a position of the signal trace. In some embodiments, the determination of the one or more displacement values comprises integrating the velocities of the velocity trace. In some embodiments, the features of the plurality of poles further comprise the radius, the angle, the velocity, complex plane distance between the plurality of poles, change in velocity between the plurality of poles, change in inertial velocity between the one or more of poles, or momentum of the plurality of poles, or any combination thereof. In some embodiments, the plurality of poles is segmented into one or more frequency groups based on frequency.

In some embodiments, generating the optimized signal trace further comprises replacing the plurality of poles of the signal trace with plurality of poles of a different frequency group. In some embodiments, the plurality of poles of the signal trace are replaced when the absolute error between one or more of the features of the plurality of poles are above a threshold value. In some embodiments, the threshold value is a value calculated by one or more of the pole selection algorithms of Table 3. In some embodiments, the poles are of opposite signs. In some embodiments, the threshold value is between 0.1 and 5.0. In some cases, the threshold value is about 0.5, 1.0, 1.5, 2.0, 2.5, 3.0, 3.5, 4.0, 4.5, 5.0, or about more than 5.0. In some embodiments, the threshold value is π/3, and the poles are of opposite signs.

In another aspect, disclosed herein are systems for determining a time-dependent position of a tissue in response to a stimulus, the systems comprising a processor comprising executable instructions stored thereon which when executed are configured to: (a) receive ultrasound data, wherein the ultrasound data is derived from an ultrasound waveform reflected from the tissue; (b) calculate from the ultrasound data a covariance matrix from a group of depths and for a plurality of pulse periods of the ultrasound data; (c) calculate one or more eigenvectors and associated eigenvalues of the covariance matrix; and (d) output a first trace of a position of the tissue associated with a content of a principal eigenvector of the one or more eigenvector.

In some embodiments, the processor is further configured to: (i) analyze a frequency content of the ultrasound data across the plurality of pulse periods; and (ii) calculate a second trace of a position of the tissue based at least in part on the frequency content.

In some embodiments, the processor is further configured to output an indication of a disease state in response to the second trace of the position of the tissue. In some embodiments, the processor is further configured to output an indication of a health state in response to the second trace of the position of the tissue. In some embodiments, the processor is further configured to output an indication of an undetermined state of the tissue in response to the second trace of the position of the tissue.

In some embodiments, the systems further comprise a pneumatic otoscope. In some embodiments, the processor is operatively connected to the pneumatic otoscope.

In some embodiments, the systems further comprise a capacitive micromachined ultrasound transducer. In some embodiments, the processor is operatively connected to the capacitive micromachined ultrasound transducer. In some embodiments, the capacitive micromachined ultrasound transducer is disposed within an otoscope.

In some embodiments, the plurality of pulse periods is sequential. In some embodiments, the tissue is a tympanic membrane. In some embodiments, the processor is further configured to output an indication of a disease state in response to the first trace of the position of the tissue. In some embodiments, the processor is further configured to output an indication of a health state in response to the first trace of the position of the tissue. In some embodiments, the processor is further configured to output an indication of an undetermined state of the tissue in response to the first trace of the position of the tissue.

In some embodiments, each pulse period of the plurality of pulse periods is associated with a related covariance matrix from a plurality of related covariance matrices. In some embodiments, the systems further comprise calculating a set of displacement vectors for the plurality of related covariance matrices. In some embodiments, the plurality of related covariance matrices is associated with pulse periods across contiguous adjacent depths of the ultrasound data. In some embodiments, the set of displacement vectors for the plurality of related covariance matrices are calculated based on a target quality signal ratio. In some embodiments, the target quality signal ratio is a value between 0.00 and 1.00. In some embodiments, the processor is further configured to stitch together segments of the set of displacement vectors to generate a time trace of the position of the tissue. In some embodiments, the time trace of the position of the tissue is the first trace.

In some embodiments, the processor of the systems is further configured to: (i) analyze one or more eigenvectors of the covariance matrix, wherein the one or more eigenvectors each comprise an orbital rotation associated with a phase of the ultrasound data: (ii) apply a bias to each of the one or more eigenvectors such that the eigenvector circles an origin in phase space; and (iii) calculate a second trace of the position of the tissues using a resulting biased eigenvector. In some embodiments, the processor is further configured to analyze the one or more eigenvectors by analyzing membrane motion information of the one or more eigenvectors.

In some embodiments, the processor is further configured to repeat the analysis in 10 ms steps in an adjustable 20 ms analysis window. In some embodiments, the processor is further configured to analyze the one or more eigenvectors by analyzing depth range values of the one or more eigenvectors. In some embodiments, the processor is further configured to analyze the one or more eigenvectors by generating a set of principal eigenvectors at each 10 ms step. In some embodiments, the processor is further configured to analyze the one or more eigenvectors by generating a set of principal eigenvectors within each 20 ms analysis window.

In some embodiments, the stimulus is a pneumatic excitation. In some embodiments, the pneumatic excitation is an air puff.

In some embodiments, the processor is further configured to: (i) determine one or more regions where the first trace comprises a non-physical tissue position or movement; and (ii) substitute at least a portion of the second trace for the first trace in the one or more regions. In some embodiments, the processor is further configured to analyze the frequency content of the complex demodulation of the ultrasound data by applying autoregression. In some embodiments, the autoregression is a third order autoregression. In some embodiments, the autoregression is higher than a third order autoregression.

In another aspect, disclosed herein are systems for improving accuracy of a time-dependent position determination of a tissue in response to a stimulus, the systems comprising a processor comprising executable instructions stored thereon which when executed are configured to: apply autoregression to a signal trace to generate a plurality of poles; eliminate or select a pole of the plurality of poles using a feature of the plurality of poles; and generate an improved signal trace of a position of the tissue from remaining or selected poles of the plurality of poles.

In some embodiments, the processor is further configured to apply the autoregression to one or more points of the signal trace. In some embodiments, the autoregression is a third order autoregression. In some embodiments, the autoregression is higher than a third order autoregression. In some embodiments, the order of the autoregression corresponds with the number of frequency peaks of the signal trace that are separated. In some embodiments, the signal trace comprises a covariance motion detection trace. In some embodiments, the autoregression is applied to one or more eigenvectors of the covariance motion detection trace.

In some embodiments, the processor of the systems is further configured to estimate a mean frequency of the signal trace using an angle value between each of the plurality of poles and a center of a complex unit circle of a complex plane. In some embodiments, the processor is further configured to calculate the angle value by taking an inverse tangent over the imaginary component of the pole of the real component of the pole. In some embodiments, the processor is further configured to estimate a variance of the signal trace using a radius value between each of the plurality of poles and a center of a complex unit circle of a complex plane. In some embodiments, the processor is further configured to estimate a velocity of a frequency of the signal trace using a radius value between each of the plurality of poles and a center of a complex unit circle of a complex plane. In some embodiments, the processor is further configured to plot a velocity trace over time of the signal trace by plotting the angle between the plurality of poles and a center of a complex unit circle of a complex plane. In some embodiments, the processor is further configured to calculate one or more displacement values between a position of the velocity trace and a position of the signal trace. In some embodiments, the processor is further configured to calculate the one or more displacement values by integrating the velocities of the velocity trace.

In some embodiments of the systems, the features of the plurality of poles further comprise the radius, the angle, the velocity, complex plane distance between the plurality of poles, change in velocity between the plurality of poles, change in inertial velocity between the one or more of poles, or momentum of the plurality of poles, or any combination thereof. In some embodiments, the processor is further configured to segment plurality of poles into one or more frequency groups based on frequency. In some embodiments, the processor is further configured to generate the optimized signal trace by replacing the plurality of poles of the signal trace with plurality of poles of a different frequency group. In some embodiments, the processor is further configured to replace plurality of poles of the signal trace when the absolute error between one or more of the features of the plurality of poles are above a threshold value. In some embodiments, the threshold value is a value calculated by one or more of the pole selection algorithms of Table 3. In some embodiments, the poles are of opposite signs. In some embodiments, the threshold value is between 0.1 and 5.0. In some cases, the threshold value is about 0.5, 1.0, 1.5, 2.0, 2.5, 3.0, 3.5, 4.0, 4.5, 5.0, or about more than 5.0. In some embodiments, the threshold value is π/3, and the poles are of opposite signs.

In another aspect, disclosed herein are systems for improving a time-dependent position determination, the systems comprising a covariance analysis model comprising executable instructions which when executed by a processor are configured to: (a) derive one or more covariance matrices from ultrasound data. (b) calculate one or more eigenvectors of the one or more covariance matrices, and (c) generate a trace from the one or more eigenvectors; one or more autoregressions configured to generate plurality of poles: an antialiasing model comprising executable instructions which when executed by a processor are configured to select one or more areas of the trace that deviates from a phase track: a signal trace model comprising executable instructions which when executed by a processor are configured to: (d) eliminate one or more poles of the one or more areas of the trace selected by the antiailiasing model, wherein the signal trace model is configured to eliminate the one or more poles based at least in part on an effect of one or more features of the one or more poles, and (e) select one or more replacement poles of one or more alternative signal traces to replace the one or more poles eliminated in (d), wherein the signal trace model is configured to select the one or more replacement poles based at least in part on an effect of one or more features of the one or more replacement poles; and an output generator configured to generate an improved signal trace of a position of the tissue from the one or more replacement poles output by the antiailiasing model.

In some embodiments, the one or more features comprise radius of the one or more poles to a center of a complex unit circle, angle between the one or more poles and the center of a complex unit circle, velocity of the angle between the one or more poles, complex plane distance between the one or more poles, change in velocity between the one or more poles, change in inertial velocity between the one or more of poles, or momentum of the one or more poles, or any combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a schematic of the pulse ultrasound wave propagation of a tone burst.

FIG. 2 illustrates an example of ultrasound data spectral analysis of arterial blood flow: Panel (A) shows an example of the echo amplitude from moving targets (blood cells) versus depth on the vertical axis, and time on the horizontal axis. Panel (B) shows an ultrasound data spectral analysis of blood flow in the proximal right middle cerebral artery (RMCA) with blood velocity in cm/s on the right vertical axis and time on the horizontal axis totaling 4 seconds duration. Panel (C) shows the intersection of an ultrasound beam with a tissue concurrently with the Right middle cerebral artery (RMCA), the right anterior cerebral artery (RACA), and the left anterior cerebral artery (LACA).

FIG. 3 illustrates ultrasound radiofrequency data from a tympanic membrane. Panel (A) shows radiofrequency (RF) echo data from a tympanic membrane (in human) where the vertical scale Depth=0 mm corresponds to 8.5 mm from the transducer, making the TM signal at approximately 13 to 15 mm depth. Panel (B) shows a magnified version of Panel (A) in the region indicated by an arrow. Panel (C) shows a magnified version of Panel (B) in the region indicated by an arrow.

FIG. 4 illustrates an enlarged version of Panel (C) of FIG. 3 with three regions highlighted and three horizontal sets of lines drawn, each line being about 20 ms in length.

FIG. 5 illustrates deconstructions of regions 1, 2 and 3 in FIG. 4 into a group of 80 overlapped phase tracks in Panels (a), (b), and (c), and illustrates the signal amplitude of regions 1, 2, and 3 in FIG. 4 in panels (d), (e), and (f).

FIG. 6 illustrates an instance of a non-physical phase behavior that may occur with tracking the tympanic membrane together with an example of unpacking and correcting it.

FIG. 7 illustrates one practical example of the discontinuity generated in the covariance technique using blood flow data.

FIG. 8 illustrates the set D of vectors containing positional change during each covariance calculation time interval in Panel (a), illustrates the curves from Panel (a) with magnification and presents the fit they have with each other in their overlapping regions in Panel (b), and illustrates the segments of Panel (b) stitched together end-to-end and revealing the tympanic motion in Panel (c).

FIG. 9 illustrates a phase profile plotted on the backdrop of the original RF ultrasound data.

FIG. 10 illustrates and example of quality Metric M, calculated across 20 ms wide windows which are spaced (overlapping 50%) in 10 ms increments for a total of 1 second duration.

FIG. 11 illustrates a zoomed in example of the covariance trace following an RF phase track of the TM similar to FIG. 9.

FIG. 12 illustrates that the covariance trace remains faithful to the phase track RF data except in two places (Time≈0.18 s and Time≈0.62).

FIG. 13 illustrates an example of a second order AR model to estimate the frequency spectrum of a signal.

FIG. 14 illustrates an example of using a first order AR model to estimate the frequency spectrum of a signal.

FIG. 15 illustrates the three poles that result if a third order AR model is applied to the first eigenvector of the covariance originally shown in FIG. 11 and FIG. 12.

FIG. 16 illustrates a plot of the pole angle over time both in velocity and radians. Pole angle is calculated by taking the inverse tangent over the imaginary component of a given pole over the real component of a given pole.

FIG. 17 illustrates a plot of the pole velocity and position for all pulse periods. Once the velocity is calculated for all pulse periods, it can be integrated over time to get the displacement of the TM.

FIG. 18 illustrates a comparison of the covariance method with pole 1 of the third order AR model.

FIG. 19 illustrates a visual outline of the process of adjusting the covariance trace based on an AR analysis.

FIG. 20 illustrates an example implementation of the antialiasing (hybrid) algorithm.

FIG. 21 illustrates a histogram of the absolute values of the error between the AR and covariance velocities.

FIG. 22 illustrates a result of the antialiasing procedure.

FIG. 23A, FIG. 23 B, and FIG. 23C illustrate regions where the pole 1 trace shows a discontinuity over three different ranges of pulse periods, 251-500, 751-1000, and 1001-1250, respectively.

FIG. 24A, FIG. 24B, FIG. 24C, FIG. 24D, and FIG. 24E illustrate five examples of implementation of the AR pole selection algorithms described above.

FIG. 25 illustrates a computer system that is programmed or otherwise configured to implement methods provided herein.

FIG. 26 is a flow chart of an example method of determining a time-dependent position of a tissue in response to a stimulus, in accordance with some embodiments.

FIG. 27 is a flow chart of an example method of improving accuracy of a time-dependent position determination of a tissue in response to a stimulus, in accordance with some embodiments.

FIG. 28 is a flow is a flow chart of an example system for improving a time-dependent position determination, in accordance with some embodiments.

FIG. 29A and FIG. 29B illustrate a side section view and a front section view of a speculum of an otoscope disposed within an ear respectively, in accordance with some embodiments.

FIG. 30A illustrates a side section view of a speculum and FIG. 30B illustrates a front section view of a tip of a speculum, in accordance with some embodiments.

FIG. 31 is a flow chart of an example method of determining a time-dependent position of a tissue in response to a stimulus incorporating an otoscope, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Disclosed herein are methods and systems for air-coupled ultrasound of the tympanic membrane. Methods and systems disclosed herein may improve determination of a position of a tympanic membrane during or near in time to the application of a pneumatic stimulus. Disclosed herein are a transducer and ultrasound system. Ultrasound signal may be measured from the medial end of the external ear canal. The ultrasound data may improve diagnosis of ear infection and systematize the collection and analysis of diagnostic information for assessing ear infection.

Methods and systems disclosed herein provide at least some of the following advantages. Tissue clutter removal is absent or nearly absent, where tissue is the quarry rather than the confounder of signals of interest. The approach to the target is through air in the external ear canal and does not arrive at the target via use of ultrasound coupling gel and travel through intervening tissue. The pulse-ultrasound transmitter/sensor may be a capacitive micromachined ultrasound transducer (CMUT).

The methods and systems disclosed herein may be used in combination with for example devices and methods to characterize a ductile membrane, surface, and sub-surface properties such as those described in commonly owned U.S. Pat. No. 7,771,356 and U.S. Patent Publication Nos. 2019/0365292, 2018/0310917, and 2017/0014053, each of which is incorporated by reference in their entireties. Further, machine learning methods and systems disclosed in commonly owned U.S. Patent Publication No. 2020/0286227 may also be used in combination with the methods and systems of the present disclosure, which is incorporated by reference in its entirety.

The methods and systems disclosed herein may be used in combination with for example air coupled capacitive micromachined ultrasound transducers and methods of use and manufacture thereof as disclosed in commonly owned U.S. Patent Publication No. 2021/0145406, which is incorporated by reference in its entirety.

The methods and systems as disclosed herein may be used in combination with for example devices and methods to transmit optical illumination such as those described in commonly owned U.S. Patent Publication 2020/0107813, which is incorporated by reference in its entirety.

The methods and systems disclosed herein may be used to characterize a number of biological tissues to provide a variety of diagnostic information. A biological tissue may comprise a patient organ. A speculum may be disposed within a bodily cavity to characterize a patient tissue. A patient organ or bodily cavity may comprise for example: a muscle, a tendon, a ligament, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, a testicle, a prostate, a heart, an artery, a vein, a spleen, a gland, a brain, a spinal cord, a nerve, etc., to name a few.

The methods and systems disclosed herein may be used to characterize a tympanic membrane. For example, a membrane may be characterized to determine a condition of an ear, such as acute otitis media (AOM). A characterization that an ear exhibits AOM may include detection of the presence of effusion and characterization of the type of effusion as one of serous, mucoid, purulent, or combinations of these. In AOM, the middle ear effusion (MEE) may be induced by infective agents and may be thin or serous with viral infection and thicker and purulent with bacterial infection. Accordingly, determining various properties of a fluid adjacent a tympanic membrane may provide information which may be used to characterize a membrane.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

### Example Ultrasound for Blood Flow

Ultrasound may be used in medical applications to understand hemodynamics and/or to survey for pathological vascular conditions which drive blood flow behavior away from expected regions with normal limits. One example is the detection of higher-than-normal blood flow velocity in an artery. Higher than normal flow may indicate a narrowing of the vessel due to a lesion which reduces the effective area of blood flow. A higher velocity within the vessel may be a warning sign for impending crisis such as heart attack or stroke. Further, higher velocity in a vessel may be an indication underpinning diagnosis and patient management for therapy.

In an example approach for processing pulse ultrasound measurements of blood flow signals, complex demodulation of the RF echo from each pulse period may be performed so that the reflected signal characteristics may be observed as a “base band” signal centered on DC. In this example, the frequency content across a collection of pulse periods may be proportional to blood flow velocity.

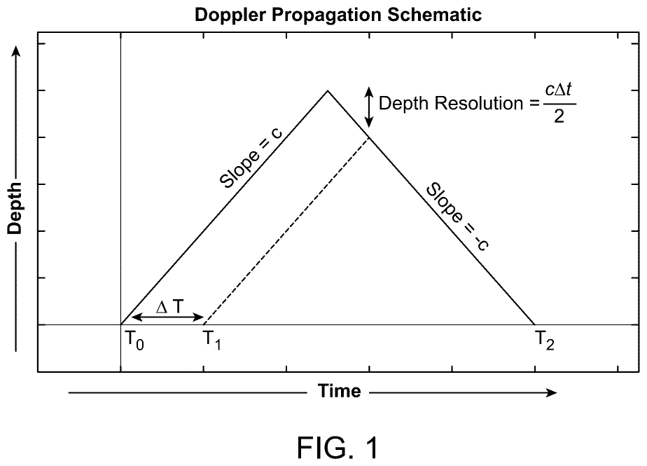

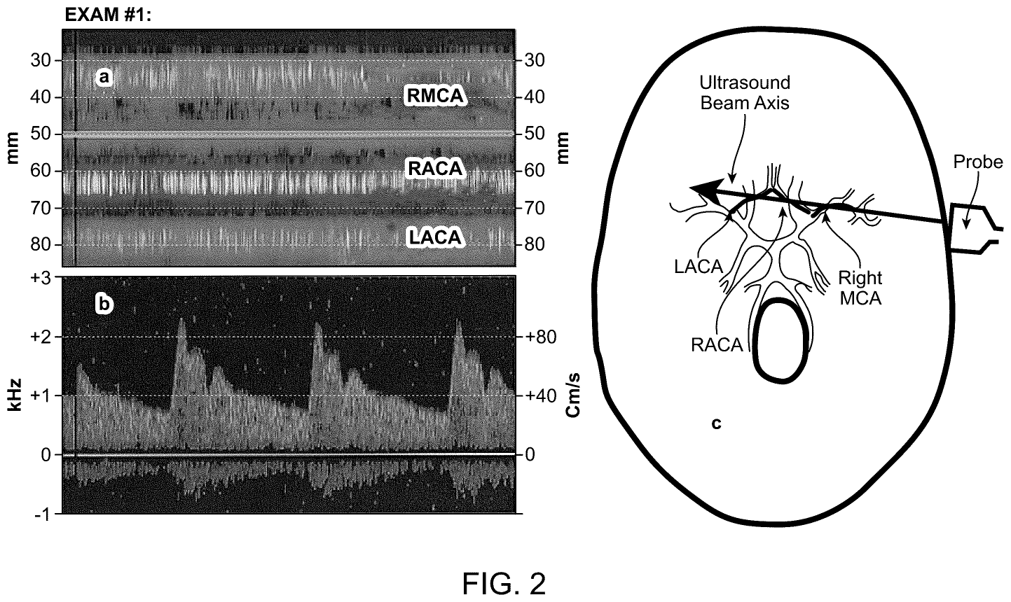

A visual summary of signal processing for pulse ultrasound of this nature is shown in FIG. 1 and FIG. 2. FIG. 1 schematically depicts the physical propagation of one ultrasound pulse into tissue and the returned echo depicting reflections, at any specific time, from a resolution cell or gate centered on a particular echo depth. The width of the resolution gate is cΔt/2, where Δt is the duration of the pulse burst emanated from the transducer. This resolution parameter is associated with the depth range of scatterers that will contribute to the echo amplitude received at the transducer at any time after the pulse burst leaves the transducer.

FIG. 1 shows a schematic of the pulse ultrasound propagation of a tone burst. A tone burst is a building block of pulse ultrasound. As shown, the leading edge is at T₀ in time, and trailing edge launched at T₁, with duration Δt=T₁−T₀. The positive sloped solid line and the positive sloped dashed line are, respectively, the leading and trailing edges of the ultrasound tone burst at any given fixed time on the horizontal axis. These lines illustrate that in the round-trip consideration, the resolution of the tone burst, called the ultrasound data sample volume size, is cΔt/2. At time T₂, there are thus echoes arriving from a range of depths spanning this sample volume size.

This activity described by this image is re-capitulated with every pulse period of an ultrasound data set and represents one vertical line among thousands in each of the motion vs time (M-mode) displays.

Panel (a) of FIG. 2 shows an example of the echo amplitude from moving targets (blood cells) versus depth on the vertical axis, and time on the horizontal axis. The vertical axis ranges from 25 to 85 mm depth, and the horizontal axis is 4 seconds in duration, and indicates a Power M-mode ultrasound analysis of blood flowing in the middle and anterior cerebral arteries supplying the brain. Panel (b) of FIG. 2 shows ultrasound spectral analysis of blood flow in the proximal RMCA (of Panel (a)). In this example, there is a high-pass filter with cutoff at about 7 cm/s in the processing chain. This filter has removed reflections from brain tissue which are about 1000 times larger than the blood flow signals. Without this “tissue clutter” removal, construction of Panel (a) may not be possible because the brain tissue signal may dominate analysis of blood flow average velocity at a given time. The ultrasound beam main axis is depicted in Panel (c) of FIG. 2, showing intersection concurrently with the Right middle cerebral artery (RMCA), the right anterior cerebral artery (RACA), and the left anterior cerebral artery (LACA). These intersection regions present echoes from blood flow that are shown in Panel (a) of FIG. 2. The blood velocity of Panel (a) is calculated from the technique of autocorrelation and is different from the position calculation in the following description employing covariance and autoregression.

### Ultrasound Measurement of the Tympanic Membrane

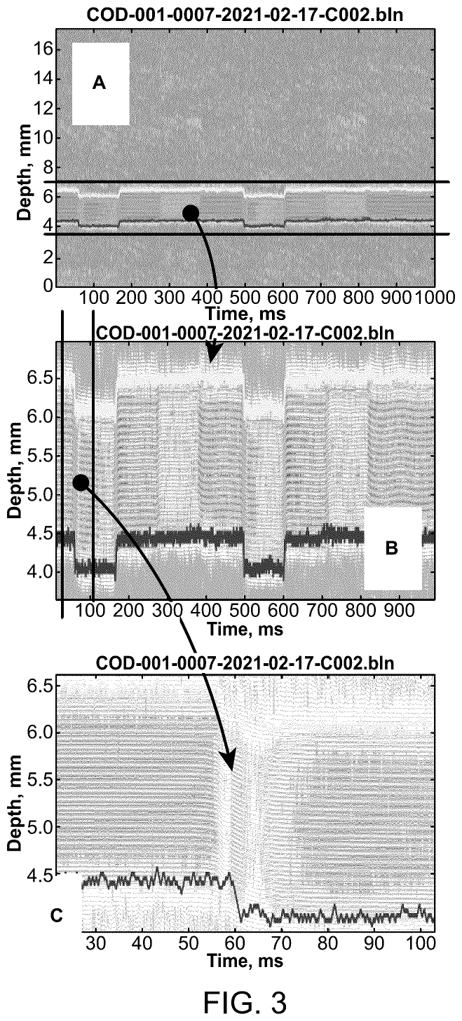

FIG. 3 shows ultrasound radiofrequency data from a tympanic membrane. Panel (A) of FIG. 3 shows RF echo data from a tympanic membrane (in a human) where the vertical scale Depth=0 mm corresponds to 8.5 mm from the transducer, making the TM signal at approximately 13 to 15 mm depth. The ultrasound pulse has 21 cycles. Note the bottom of the image is closest to the transducer. The image between the two horizontal lines in Panel (A) of FIG. 3 is magnified to generate Panel (B) of FIG. 3, and the image between the two vertical lines in Panel (B) of FIG. 3 is further magnified to generate Panel (C) of FIG. 3. The absence of RF data in the horizontal region around 80 ms in Panel (C) of FIG. 3 is due to diminished reflection energy from the TM. This diminished energy makes tracking of the TM position more difficult. Accordingly, because of the diminished signal in this region, it may be useful to take all the reflection data into account in determining TM position. The covariance and autoregression techniques described herein address this signal region at least in part.

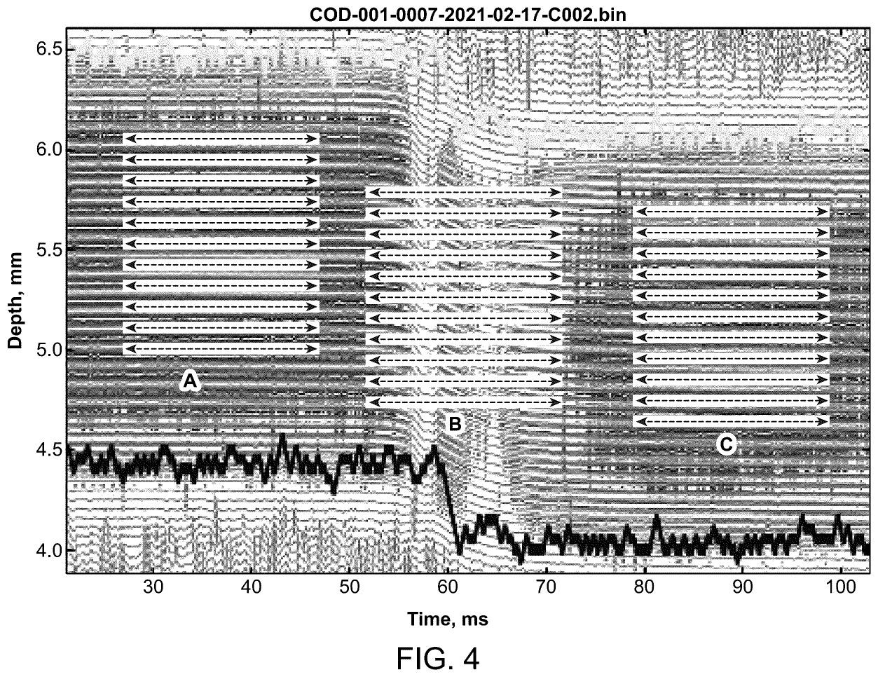

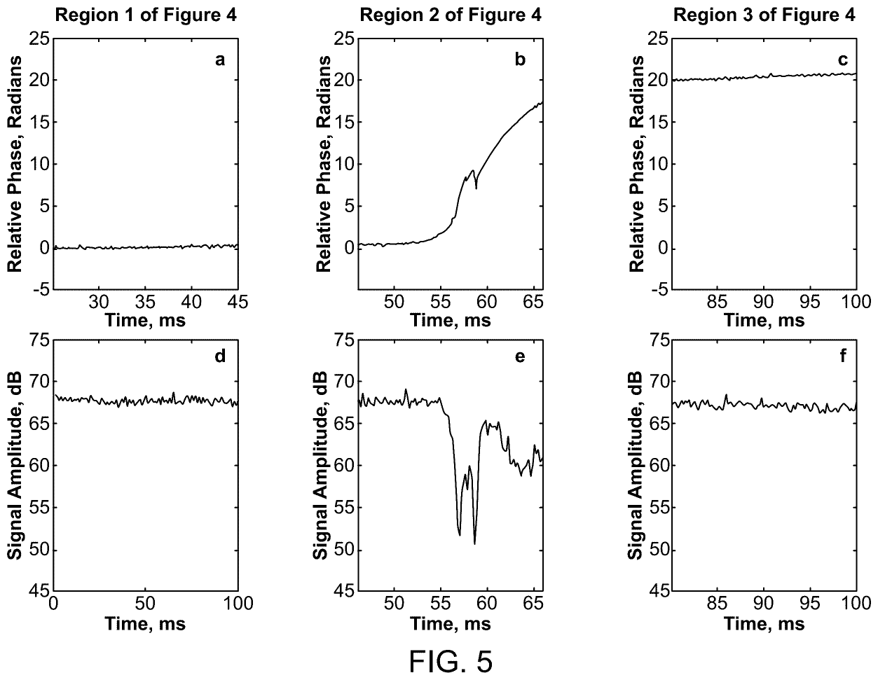

FIG. 4 shows an enlarged version of Panel (C) of FIG. 3. As shown, three horizontal sets of lines are drawn, each line being about 20 ms in length. The three sets are placed at different non-overlapping regions of the TM echo reflection. Each set of 10 lines is an example instance for sampling the complex RF echo (via Hilbert transform) and determining a covariance matrix from the summed outer product of each of the ten complex echo vectors.

FIG. 5 show deconstructions of regions 1, 2 and 3 in FIG. 4 into a group of 80 overlapped phase tracks in Panels (a), (b), and (c) respectively. Each track is comprised of a value from each of M pulse periods (for example, M=100 at 5 kHz pulse repetition frequency, or a time span of 100/5000=20 ms). In this group of 80 tracks, there is one track per gate depth or a/d sample increment.

Panels (a), (b), and (c) of FIG. 5 have 80 phase tracks drawn on top of each other and show that the source of this data, the RF echo, is very consistent in its phase.

Panels (d), (e), and (f) of FIG. 5 show the signal amplitude of regions 1, 2, and 3 in FIG. 4. In Panel (b) of FIG. 5, there is a singular jaggedness in the phase progression. This is further illuminated by noting the signal energy level in Panels (d), (e), and (f) of FIG. 5. The energy has a noticeable drop of 15 to 20 dB in the region of the jagged region in Panel (b) of FIG. 5. This jaggedness in the phase spectrum may create challenges if the unwrapped phase from a Hilbert transform is used to extract the motion dynamics of the scattering target, in this case the tympanic membrane, without other correction.

### Methods for Determining a Time-Dependent Position of a Tissue in Response to a Stimulus

Disclosed herein are methods and systems for determining a time-dependent position of a tissue in response to a stimulus. The tissue can be a biological tissue. The tissue can be plant, a food, an animal, or a human. The tissue can be a human tissue, such as an organ. The tissue can be a smooth tissue organ or a musculoskeletal organ. The tissue can be, for example, a heart, a liver, a spleen, an organ of the ear, a gallbladder, a pancreas, kidneys, a bladder, a uterus or ovaries, a prostate, glands such as thyroid and parathyroid glands, blood vessels, ocular organs, appendix, stomach and intestines, or any other organs. The tissue can be a tympanic membrane. The stimulus can be a physical stimulus. The stimulus can be a pressure stimulus, an electrical stimulus, an electromagnetic stimulus, a physical contact stimulus, a liquid injection stimulus, or sensory stimulus, or another type of stimulus. The stimulus can be a pressure changing stimulus.

In some cases, the method can comprise receiving ultrasound data. In other embodiments, the method can comprise receiving another type of electromagnetic data. The ultrasound data can be derived from an ultrasound waveform reflected from the tissue. In other embodiments, ultrasound data can be derived from one or more fragments of an ultrasound waveform reflected from the tissue. The method can comprise calculating from the ultrasound data a covariance matrix. The covariance data can be a function of depth for a plurality of pulse periods of the ultrasound data. The covariance data can be a function of depth for about two pulse periods, three pulse periods, four pulse periods, five pulse periods, about ten pulse periods, about fifteen pulse periods, about twenty pulse periods, about twenty-five pulse periods, about fifty pulse periods, about one hundred pulse periods, about two hundred pulse periods, about three hundred pulse periods, about four hundred pulse periods, about five hundred pulse periods, about one thousand pulse periods, about two thousand pulse periods, about three thousand pulse periods, about four thousand pulse periods, about five thousand pulse periods, or more than about five thousand pulse periods. In some cases, the number of pulse periods can be between 4500 and 5000 pulse periods. In some cases, the number of pulse periods can be 4800 pulse periods. The method can further comprise calculating one or more eigenvectors and associated eigenvalues of the covariance matrix. In some cases, the method can comprise calculating one eigenvector, two eigenvectors, three eigenvectors, four eigenvectors, five eigenvectors, six eigenvectors, seven eigenvectors, eight eigenvectors, nine eigenvectors, ten eigenvectors, eleven eigenvectors, twelve eigenvectors, thirteen eigenvectors, fourteen eigenvectors, fifteen eigenvectors, sixteen eigenvectors, seventeen eigenvectors, eighteen eigenvectors, nineteen eigenvectors, twenty eigenvectors, twenty-one eigenvectors, twenty-two eigenvectors, twenty-three eigenvectors, twenty-four eigenvectors, twenty-five eigenvectors, twenty-six eigenvectors, twenty-seven eigenvectors, twenty-eight eigenvectors, twenty-nine eigenvectors, thirty eigenvectors, thirty-one eigenvectors, thirty-two eigenvectors, thirty-three eigenvectors, thirty-four eigenvectors, thirty-five eigenvectors, thirty-six eigenvectors, thirty-seven eigenvectors, thirty-eight eigenvectors, thirty-nine eigenvectors, forty eigenvectors, forty-one eigenvectors, forty-two eigenvectors, forty-three eigenvectors, forty-four eigenvectors, forty-five eigenvectors, forty-six eigenvectors, forty-seven eigenvectors, forty-eight eigenvectors, forty-nine eigenvectors, fifty eigenvectors, or more than fifty eigenvectors. The method can further comprise determining a first trace of a position of the tissue associated with a content of a principal eigenvector of the one or more eigenvectors.

In some cases, the methods can further comprise determining a time-dependent position of a tissue in response to a stimulus by analyzing a frequency content of the ultrasound data across a plurality of pulse periods. In some cases, the frequency content can be a trace. In some cases, the method can further comprise calculating a second trace of a position of the tissue based at least in part on the frequency content. In some cases, the frequency content can be a measurement of the frequency. In some cases, the frequency content can be a change in frequency or velocity of the frequency.

In some cases, the methods can further comprise outputting an indication of a disease state of the tissue. In some cases, the indication can be in response to a trace. In some cases, the indication can be in response to the second trace of the position of the tissue. In other embodiments, the methods can further comprise outputting an indication of a health state of the tissue. In some cases, the indication can be in response to the second trace of the position of the tissue. In other embodiments, the methods can further comprise outputting an indication of an undetermined state of the tissue. In some cases, the indication can be in response to the second trace of the position of the tissue. In some cases, the indication can be in response to a first trace of the tissue. In some cases, the indication can be in response to a third trace of the tissue.

In some cases, the plurality of pulse periods can be sequential. In other cases, the plurality of pulse periods can be non-sequential. In some cases, the tissue can be an organ of the ear, such as the tympanic membrane, the bones of the ear, the eustachian tube, the cochlea, the oval window, or another organ of the ear. In some cases, the tissue can be another human organ such as the heart, liver, kidneys, stomach, eye, brain, or another organ.

In some cases, the methods can further comprise outputting an indication of a disease state of the tissue. In some cases, the indication can be in response to a trace. In some cases, the indication can be in response to the first trace of the position of the tissue. In other embodiments, the methods can further comprise outputting an indication of a health state of the tissue. In some cases, the indication can be in response to the first trace of the position of the tissue. In other embodiments, the methods can further comprise outputting an indication of an undetermined state of the tissue. In some cases, the indication can be in response to the first trace of the position of the tissue. In some cases, the indication can be in response to a second trace of the tissue. In some cases, the indication can be in response to a third trace of the tissue.

In some cases, each pulse period of the plurality of pulse periods can be associated with a related covariance matrix from a plurality of related covariance matrices. In some cases, the plurality of covariance matrices can comprise two covariance matrices, three covariance matrices, four covariance matrices, five covariance matrices, six covariance matrices, seven covariance matrices, eight covariance matrices, nine covariance matrices, ten covariance matrices, eleven covariance matrices, twelve covariance matrices, thirteen covariance matrices, fourteen covariance matrices, fifteen covariance matrices, sixteen covariance matrices, seventeen covariance matrices, eighteen covariance matrices, nineteen covariance matrices, twenty covariance matrices, twenty-one covariance matrices, twenty-two covariance matrices, twenty-three covariance matrices, twenty-four covariance matrices, twenty-five covariance matrices, twenty-six covariance matrices, twenty-seven covariance matrices, twenty-eight covariance matrices, twenty-nine covariance matrices, thirty covariance matrices, thirty-one covariance matrices, thirty-two covariance matrices, thirty-three covariance matrices, thirty-four covariance matrices, thirty-five covariance matrices, thirty-six covariance matrices, thirty-seven covariance matrices, thirty-eight covariance matrices, thirty-nine covariance matrices, forty covariance matrices, forty-one covariance matrices, forty-two covariance matrices, forty-three covariance matrices, forty-four covariance matrices, forty-five covariance matrices, forty-six covariance matrices, forty-seven covariance matrices, forty-eight covariance matrices, forty-nine covariance matrices, fifty covariance matrices, or more than fifty covariance matrices.

In some cases, the methods can further comprise calculating a set of displacement vectors for the plurality of related covariance matrices. In some cases, the plurality of related covariance matrices can be associated with pulse periods across contiguous adjacent depths of ultrasound data. In some cases, analysis of the covariance matrix across the phase track can reveal principal components of the tympanic membrane motion. This technique can be advantageous to help remedy a low energy jagged ultrasound signal, as shown in Panel (b) of FIG. 5. The covariance matrix may be constructed by taking the Hilbert transformed RF ultrasound samples within a region of ultrasound data. The region can be a clear echo region. The region may also be consistent with the length of the pulse period of the ultrasound data. The covariance matrix can be analyzed by a Hilbert transform.

The analytic pulse echo signal that results from the Hilbert transform of a single pulse period, denoted by “k” for kth pulse period, may be expressed as:

$$H ( z , k ) = H ( m , k ) = R ( m , k ) exp ( j [ ω t + φ ( m , k ) ] )$$

where R is amplitude, ω=2πf is the carrier frequency, φ is signal phase, and z is depth and corresponds, if discretized, to the m^{th} a/d sample. The analytic pulse echo signal can be used to find elements of the covariance matrices. In some cases, the matrices can be N-by-N covariance matrices. In some cases, the covariance matrices can be from the same depth but two different pulse periods. These pulse periods can each be a known number of pulse periods from pulse period k. These pulse periods can be denoted by indices p and q. The covariance matrix summand can arise from a fixed depth, z or m. The covariance matrix can be an outer product matrix described by

$$K ( p , q , m ) = H ( m , p ) * {H}^{′} ( m , q )$$

where p={0 . . . N−1} and q={0 . . . N−1}, and the apostrophe can denote complex conjugation.

The completed covariance matrix can sum K(p, q, m) from a group of adjacent depths m={m0, m1, m2, . . . , mR}. Plugging in H from above into this covariance expression yields the following:

$$K ( p , q , m ) = R ( m , p ) ( j [ ω t + φ ( m , p ) ] \cdot R ( m , q ) exp ( - j [ ω t + ϕ ( m , q ) ] )$$

which is further simplified to:

$$K ( p , q , m ) = R ( m , p ) \cdot R ( m , q ) \cdot exp ( j [ φ ( m , p ) - φ ( m , q ) ] ) .$$

The covariance matrix arising from contributions across a series of contiguous depths spanning the specific tympanic membrane echo can be denoted by:

$$K ( p , q ) = {\sum }_{m = m 0}^{m R} R ( p , m ) \cdot R ( q , m ) \cdot exp ( j [ φ ( m , p ) - φ ( m , q ) ] )$$

which is written more simply as

$$K ( p , q ) = {\sum }_{m = m 0}^{m R} {R}_{m} ( p ) {R}_{m} ( q ) {e}^{j Δ}$$

where

$$Δ = Δ ( p , q ) = {φ}_{m} ( p ) - {φ}_{m} ( q ) .$$

and Δ can be a difference in phase sampled at one depth, m, at two different times, p and q. This can also be known as the time rate of change in phase of an ultrasound signal.

This can avoid demodulation because the carrier frequency at the start of the description of K is effectively subtracted out by multiplication of one exponential by a second which has been conjugated.

The covariance matrix may be calculated based on the above mathematical description. With the resulting matrix, the eigenvectors and eigenvalues may be denoted by:

$$K \vert {v}_{1} , {v}_{2} , {v}_{3} … {v}_{N} \vert = \vert \begin{matrix}{λ}_{1} & 0 & & 0 \\ 0 & {λ}_{2} & & \\ & & ⋱ & 0 \\ 0 & & 0 & {λ}_{N}\end{matrix} \vert \vert {v}_{1} , {v}_{2} , {v}_{3} … {v}_{N} \vert .$$

where v₁, v₂, v₃ . . . v_{N} is the set of eigenvectors and (λ₁, λ₂, λ₃ . . . ) are the eigenvalues.

In some cases, the displacement vectors for the plurality of related covariance matrices are calculated based on a target quality signal ratio. The target quality signal ratio can be a value between 0.00 and 1.00. In some cases, the target quality signal ratio can be 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.00.

In some cases, the methods can further comprise stitching together segments of the set of displacement vectors to generate a time trace of the position of the tissue. In some cases, the time trace can be the first trace. In some cases, the time trace can be the second trace. In some cases, the time trace can be the third trace.

In some cases, methods of determining a time-dependent position of a tissue in response to a stimulus can further comprise analyzing one or more eigenvectors of the covariance matrix. In some cases, the one or more eigenvectors can each comprise an orbital rotation associated with a phase of the ultrasound data. It is noted that there are many ways to express the eigenvectors and eigenvalues for a covariance operator. In some cases, the membrane motion may be generally contained within the first eigenvector. In some cases, the membrane motion may be generally contained within the second eigenvector. In some cases, the membrane motion may be generally contained within the third eigenvector. In some cases, the membrane motion may be generally contained within a different eigenvector.

In some cases, eigenvectors can have similarity of phase track behavior across many depths, as shown in Panels (a), (b), and (c) of FIG. 5. In some cases, the overall signal energy may be clustered in just a small number of eigenvectors. In some cases, the eigenvectors {v₁, v₂, v₃ . . . v_{N}} can constitute an orthonormal basis set. In some cases, the ratio of each eigenvalue to the sum of all eigenvalues can be the fraction of signal class energy represented in an associated eigenvector. In some cases, the eigenvalue energy can be contained within one eigenvector. In some cases, places where the signal is energy depleted such as in Panel (b) of FIG. 5 may show a concurrent drop in the energy fraction contained in the first eigenvector. In some cases, to quantify this, a target signal quality ratio may be expressed by:

$$M = {λ}_{1} / {\sum }_{m = 1}^{N} {λ}_{m} .$$

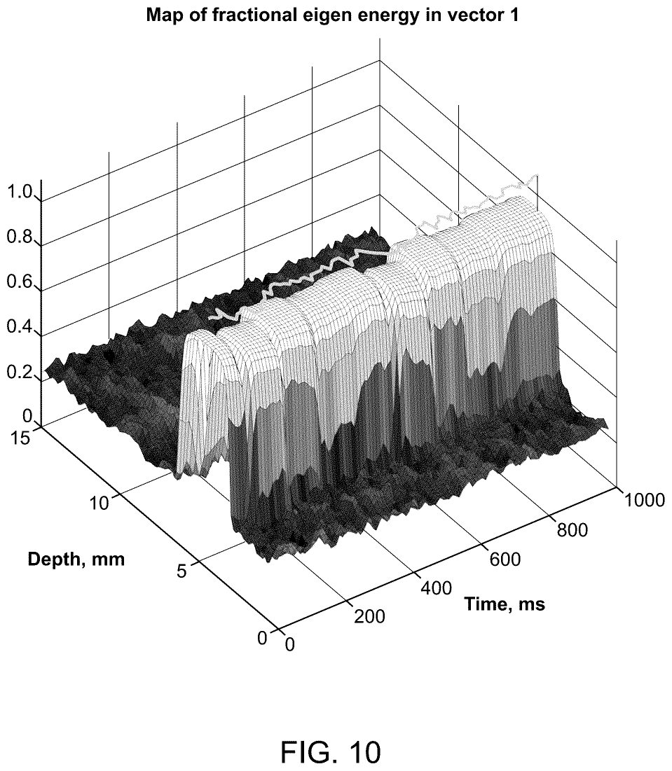

M can be evaluated at various depths along the ultrasound beam over the number of pulse periods contained in the covariance calculation. When there is no target present, M can be a value between 0 and 1. In some cases, M can have higher variance than when a target is present. These facets of the quality ratio behavior are seen, for example, in FIG. 10.

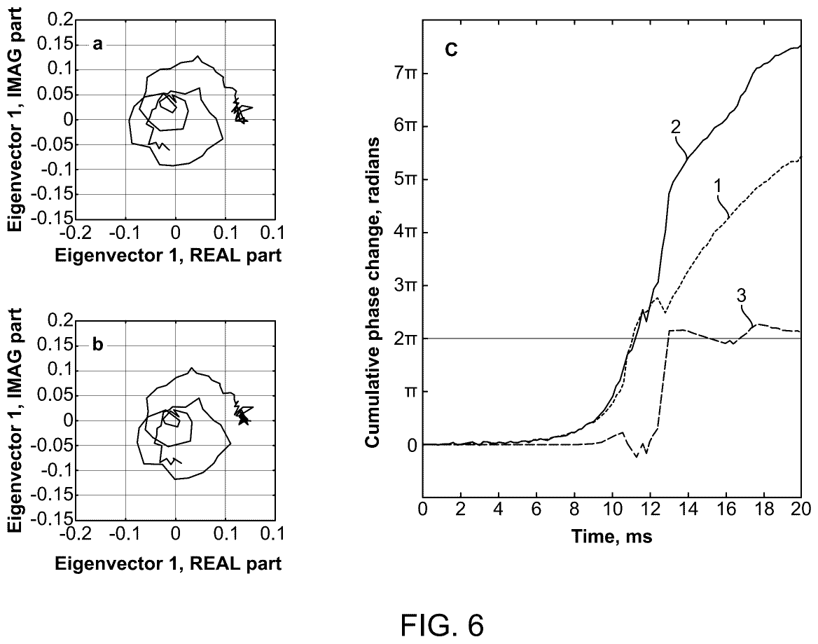

A physical model of tympanic membrane movement can show continuous circulation of phase without jumps or discontinuities. Panels (a), (b), and (c) of FIG. 6 illustrate an example of a non-physical phase behavior that may occur with tracking the tympanic membrane together with an example of unpacking and correcting it.

Panel (a) of FIG. 6 shows an example of an eigenvector of the covariance from Region 2 of FIG. 4. In some cases, an eigenvector can show, for example, an amplitude drop. The observed drop may occur because the reflection strength from the tympanic membrane becomes low relative to the reflection from nearby tissue (such as the umbo) which is stationary (nonmoving) by comparison. Panel (a) of FIG. 6 shows an example of one of the orbital cycles of the path, which can be biased. Panel (b) of FIG. 6 shows the same eigenvector as in Panel (a) of FIG. 6, but includes, for example, a pre-inserted bias so that all orbits of the signal are about the complex plane origin. Panel (c) of FIG. 6 shows, for example, the cumulative phase change of the path in Panel (a) of FIG. 6 as trace 1, and the cumulative phase change of the path in Panel (b) of FIG. 6 as trace 2. The difference between traces 2 and 1 can be seen, for example in the dashed trace 3. There can be, for example a 2π difference due to the bias correction from Panel (a) of FIG. 6 to Panel (b) of FIG. 6.

In some cases, methods of determining a time-dependent position of a tissue in response to a stimulus can further comprise applying a bias to each of the one or more eigenvectors such that the eigenvector circles an origin in phase space. While adding a corrective bias to the data, such as is shown Panel (b) of FIG. 6, may be one solution to the problem of non-physical phase extractions, it may add its own artifact to the data.

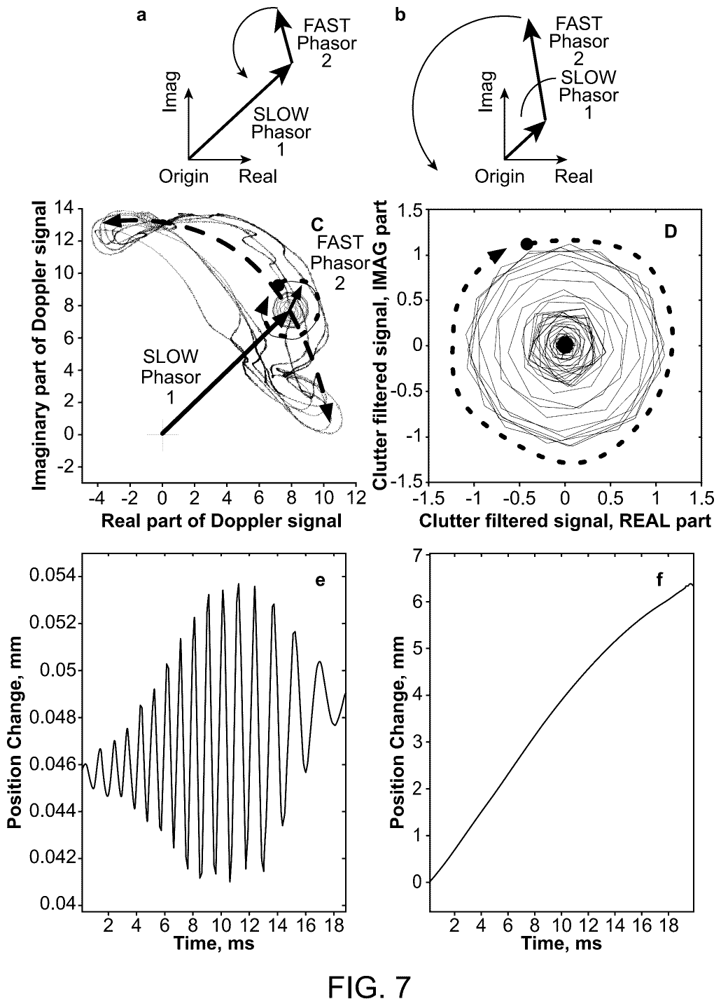

FIG. 7 shows, for example, that the ultrasound signal can be a linear sum of phasors in the complex plane. As shown, motion associated with a particular phasor may be obscured if the phasor orbital center is biased away from the origin of the complex plane. Panel (a) of FIG. 7 illustrates phasor 2, which can be the signal of interest, which can orbit away from the origin because of a bias from Phasor 1. Panel (b) of FIG. 7 illustrates that phasor 2, which can be the signal of interest, can orbit around the complex plane origin. Panel (c) of FIG. 7 illustrates an example of the two phasor behavior of Panel (a) of FIG. 7 with ultrasound signal data from brain tissue. In some cases, phasor 1 can be the slow-moving phasor, and a particulate in blood flow in the middle cerebral artery is phasor 2, which can be the fast-moving phasor. Panel (d) of FIG. 7 illustrates the example of Panel (b) of FIG. 7 with same brain/particulate data, but the SLOW brain phasor can be filtered out with a high-pass filter (clutter filter), leaving just the embolus signal as the residual phasor orbiting the complex origin. Panel (e) of FIG. 7 shows an exemplary calculation of embolus position based on data with Panel (a) of FIG. 7 type-bias, which can show inaccurately very little motion of the embolus moving in blood flow. Panel (f) of FIG. 7 can show, for example, a calculation of embolus position based on data with the character of Panel (b) of FIG. 7 and Panel (d) of FIG. 7, which can result in accuracy improvements in calculating the motion of the embolus.

As shown in Panel (e) of FIG. 5, in some examples, there can be a dip in the signal to noise in the region near the target. The target can be, for example, the tympanic membrane. Covariance analysis across the entire same region containing the target may, in some cases, diminish broadband noise and make the motion pathway and dynamics more apparent. In some cases, noise process covariance (data with no target present) results in the use of more eigenvectors to describe the process than for target covariance (data with a target present). Covariance analysis with a target present can, in some cases, result in 1 or single digit components to describe target dynamics. The principal eigenvector (with the largest eigenvalue) can, in some cases, describe target motion which has contribution from all the a/d samples that are within the target echo. In some cases, the broadband noise may tend to be separately analyzable from the signal.

In some cases, methods of determining a time-dependent position of a tissue in response to a stimulus can further comprise using a resulting biased eigenvector to calculate a second trace of the position of the tissues. In some cases, analyzing the one or more eigenvectors can further comprise analyzing membrane motion information of the one or more eigenvectors. In some cases, analyzing the one or more eigenvectors can further comprise repeating the analysis in 10 millisecond (ms) steps. In some cases, the 10 ms steps can be in an adjustable 20 ms analysis window. In some cases, analyzing the one or more eigenvectors can further comprise analyzing depth range values. In some cases, analyzing the one or more eigenvectors can further comprise creating a set of principal eigenvectors at each 10 millisecond (ms) step. In some cases, analyzing the one or more eigenvectors can further comprise creating a set of principal eigenvectors within each 20 ms analysis window.

In some examples, repeating the calculation of the principal eigenvector of the covariance analysis across the depth extent and the time extent of the signal can be shown in Panel (A) of FIG. 3. In the example shown in Panel (A) of FIG. 3, in some cases, the data stream can extend for 1 second duration during the tympanic membrane data capture. This can be, for example, about 5000 pulse periods of data over which to do a covariance analysis. In some cases, the analysis may be repeated in 10 ms steps. In some cases, the analysis may be further repeated over the 20 ms window from the early edge of the signal to the latest edge of the signal. In some cases, the analysis can be further repeated over the M₁ values central to the tympanic membrane silhouette. For example, the top 80 M₁ values at each time position in FIG. 10 relative to the silhouette of Panel (A) of FIG. 3 and Panel (B) of FIG. 3. In some cases, the stimulus can be an excitation. In some cases, the excitation can be a physical, electrical, or electromagnetic excitation. In some cases, the stimulus can be a pneumatic excitation. In some cases, the pneumatic excitation can be an air puff.

For example, the set of principal eigenvectors that can result, 1 per window position, at each of 100 windows in 10 ms increments across the 1 second time range, may be represented as

$$V = \{ {v}_{1}^{( 1 )} , {v}_{1}^{( 2 )} , {v}_{1}^{( 3 )} , … , {v}_{1}^{( N )} \} .$$

The membrane motion information contained in an eigenvector may obtained by the following expression

$${\vec{d}}_{k} = \frac{U ( ∠ {v}_{1}^{( k )} ) λ}{4 π}$$

where U is the “phase unwrap operator”. This expression can be the unwrapped eigenvector phase, factored by one wavelength of carrier frequency for every 4π radians of phase change. \vec{d} can be a vector which has one value for every pulse period included in the covariance analysis window.

Similar to the set of eigenvectors above, there is a set of displacement vectors which can be denoted by

$$D = \{ {\vec{d}}_{1} , {\vec{d}}_{2} , {\vec{d}}_{3} , … , {\vec{d}}_{N} \} .$$

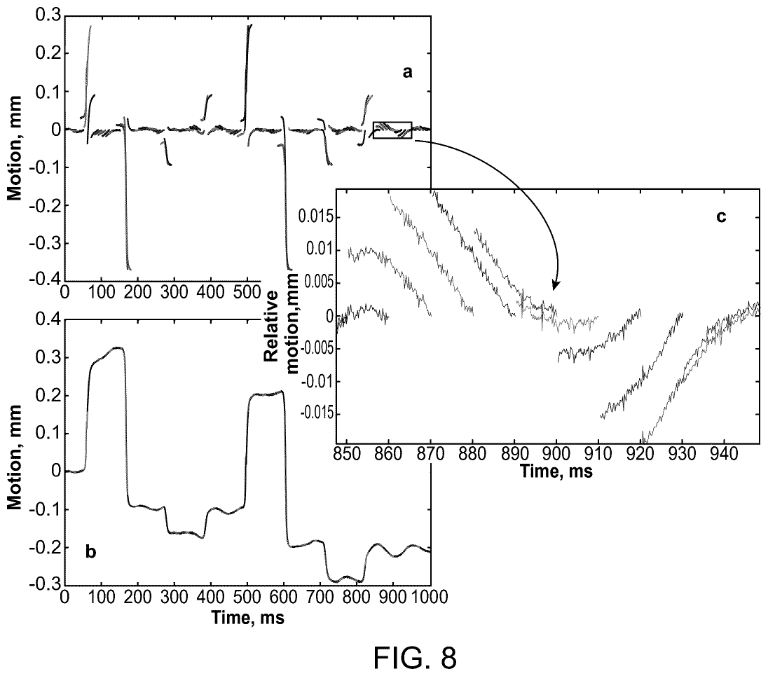

Panel (a) of FIG. 8 shows an example of the set D of vectors containing positional change during each covariance calculation time interval. These individual segments can show the relative position change made by the tympanic membrane. In some cases, they may be “stitched” to their adjacent neighbors. Panel (b) of FIG. 8 shows, for example, these curves with magnification and presents the fit they have with each other in their overlapping regions. Panel (c) of FIG. 8 shows an example of the segments stitched together end-to-end and revealing the tympanic motion.

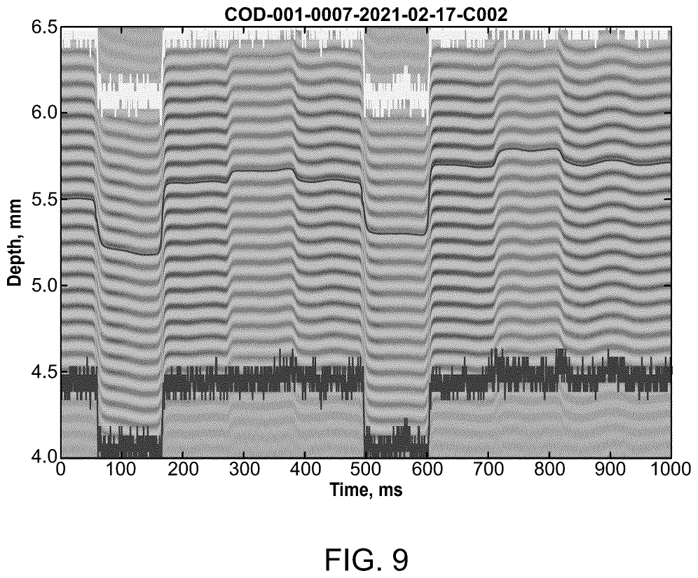

FIG. 9 illustrates the phase profile plotted, for example, on the backdrop of the original RF ultrasound data.

FIG. 10 illustrates an example of quality Metric M, calculated across 20 ms wide windows which are spaced in 10 ms increments for a total of 1 second duration. The noise background can be, for example, close to a level of 0.2, compared to the “target” which is close to 1.0 with occasional dips across the time span and across a depth span equal to the ultrasound sample volume size. The path at the top can be the position of the maxim value of M across depth, at each 10 ms increment.

### Systems for Determining a Time-Dependent Position of a Tissue in Response to a Stimulus

Disclosed herein are systems for determining a time-dependent position of a tissue in response to a stimulus. The tissue can be a biological tissue. The tissue can be plant, a food, an animal, or a human. The tissue can be a human tissue, such as an organ. The tissue can be a smooth tissue organ or a musculoskeletal organ. The tissue can be, for example, a heart, a liver, a spleen, an organ of the ear, a gallbladder, a pancreas, kidneys, a bladder, a uterus or ovaries, a prostate, glands such as thyroid and parathyroid glands, blood vessels, ocular organs, appendix, stomach and intestines, or any other organs. The tissue can be a tympanic membrane. The stimulus can be a physical stimulus. The stimulus can be a pressure stimulus, an electrical stimulus, an electromagnetic stimulus, a physical contact stimulus, a liquid injection stimulus, or sensory stimulus, or another type of stimulus. The stimulus can be a pressure changing stimulus.

The systems can comprise a processor comprising executable instructions stored thereon which, when executed, is configured to receive ultrasound data. In other embodiments, the processor is configured to receive another type of electromagnetic data. The ultrasound data can be derived from an ultrasound waveform reflected from the tissue. In other embodiments, ultrasound data can be derived from one or more fragments of an ultrasound waveform reflected from the tissue. The processor can be configured to calculate from the ultrasound data a covariance matrix. The covariance data can be a function of depth for a plurality of pulse periods of the ultrasound data. The covariance data can be a function of depth for about two pulse periods, three pulse periods, four pulse periods, five pulse periods, about ten pulse periods, about fifteen pulse periods, about twenty pulse periods, about twenty-five pulse periods, about fifty pulse periods, about one hundred pulse periods, about two hundred pulse periods, about three hundred pulse periods, about four hundred pulse periods, about five hundred pulse periods, about one thousand pulse periods, about two thousand pulse periods, about three thousand pulse periods, about four thousand pulse periods, about five thousand pulse periods, or more than about five thousand pulse periods. In some cases, the number of pulse periods can be between 4500 and 5000 pulse periods. In some cases, the number of pulse periods can be 4800 pulse periods. The processor can be configured to calculate one or more eigenvectors and associated eigenvalues of the covariance matrix. In some cases, the processor can be configured to calculate one eigenvector, two eigenvectors, three eigenvectors, four eigenvectors, five eigenvectors, six eigenvectors, seven eigenvectors, eight eigenvectors, nine eigenvectors, ten eigenvectors, eleven eigenvectors, twelve eigenvectors, thirteen eigenvectors, fourteen eigenvectors, fifteen eigenvectors, sixteen eigenvectors, seventeen eigenvectors, eighteen eigenvectors, nineteen eigenvectors, twenty eigenvectors, twenty-one eigenvectors, twenty-two eigenvectors, twenty-three eigenvectors, twenty-four eigenvectors, twenty-five eigenvectors, twenty-six eigenvectors, twenty-seven eigenvectors, twenty-eight eigenvectors, twenty-nine eigenvectors, thirty eigenvectors, thirty-one eigenvectors, thirty-two eigenvectors, thirty-three eigenvectors, thirty-four eigenvectors, thirty-five eigenvectors, thirty-six eigenvectors, thirty-seven eigenvectors, thirty-eight eigenvectors, thirty-nine eigenvectors, forty eigenvectors, forty-one eigenvectors, forty-two eigenvectors, forty-three eigenvectors, forty-four eigenvectors, forty-five eigenvectors, forty-six eigenvectors, forty-seven eigenvectors, forty-eight eigenvectors, forty-nine eigenvectors, fifty eigenvectors, or more than fifty eigenvectors. The processor can further be configured to determine a first trace of a position of the tissue associated with a content of a principal eigenvector of the one or more eigenvectors.

In some cases, the systems can further comprise a processor configured to determine a time-dependent position of a tissue in response to a stimulus by analyzing a frequency content of the ultrasound data across a plurality of pulse periods. In some cases, the frequency content can be a trace. In some cases, the processor can be configured to calculate a second trace of a position of the tissue based at least in part on the frequency content. In some cases, the frequency content can be a measurement of the frequency. In some cases, the frequency content can be a change in frequency or velocity of the frequency.

In some cases, the systems can further comprise a processor configured to output an indication of a disease state of the tissue. In some cases, the indication can be in response to a trace. In some cases, the indication can be in response to the second trace of the position of the tissue. In other embodiments, the systems can further comprise a processor configured to output an indication of a health state of the tissue. In some cases, the indication can be in response to the second trace of the position of the tissue. In other embodiments, the systems can further comprise can further comprise a processor configured to output an indication of an undetermined state of the tissue. In some cases, the indication can be in response to the second trace of the position of the tissue. In some cases, the indication can be in response to a first trace of the tissue. In some cases, the indication can be in response to a third trace of the tissue.

In some cases, the plurality of pulse periods can be sequential. In other cases, the plurality of pulse periods can be non-sequential. In some cases, the tissue can be an organ of the ear, such as the tympanic membrane, the bones of the ear, the eustachian tube, the cochlea, the oval window, or another organ of the ear. In some cases, the tissue can be another human organ such as the heart, liver, kidneys, stomach, eye, brain, or another organ.

In some cases, the systems can further comprise can further comprise a processor configured to output an indication of a disease state of the tissue. In some cases, the indication can be in response to a trace. In some cases, the indication can be in response to the first trace of the position of the tissue. In other embodiments, the systems can further comprise a processor configured to output an indication of a health state of the tissue. In some cases, the indication can be in response to the first trace of the position of the tissue. In other embodiments, the systems can further comprise a processor configured to output an indication of an undetermined state of the tissue. In some cases, the indication can be in response to the first trace of the position of the tissue. In some cases, the indication can be in response to a second trace of the tissue. In some cases, the indication can be in response to a third trace of the tissue.

In some cases, each pulse period of the plurality of pulse periods can be associated with a related covariance matrix from a plurality of related covariance matrices. In some cases, the plurality of covariance matrices can comprise two covariance matrices, three covariance matrices, four covariance matrices, five covariance matrices, six covariance matrices, seven covariance matrices, eight covariance matrices, nine covariance matrices, ten covariance matrices, eleven covariance matrices, twelve covariance matrices, thirteen covariance matrices, fourteen covariance matrices, fifteen covariance matrices, sixteen covariance matrices, seventeen covariance matrices, eighteen covariance matrices, nineteen covariance matrices, twenty covariance matrices, twenty-one covariance matrices, twenty-two covariance matrices, twenty-three covariance matrices, twenty-four covariance matrices, twenty-five covariance matrices, twenty-six covariance matrices, twenty-seven covariance matrices, twenty-eight covariance matrices, twenty-nine covariance matrices, thirty covariance matrices, thirty-one covariance matrices, thirty-two covariance matrices, thirty-three covariance matrices, thirty-four covariance matrices, thirty-five covariance matrices, thirty-six covariance matrices, thirty-seven covariance matrices, thirty-eight covariance matrices, thirty-nine covariance matrices, forty covariance matrices, forty-one covariance matrices, forty-two covariance matrices, forty-three covariance matrices, forty-four covariance matrices, forty-five covariance matrices, forty-six covariance matrices, forty-seven covariance matrices, forty-eight covariance matrices, forty-nine covariance matrices, fifty covariance matrices, or more than fifty covariance matrices.

In some cases, the systems can further comprise a processor configured to calculate a set of displacement vectors for the plurality of related covariance matrices. In some cases, the plurality of related covariance matrices can be associated with pulse periods across contiguous adjacent depths of ultrasound data. In some cases, analysis of the covariance matrix across the phase track can reveal principal components of the tympanic membrane motion. This technique can be advantageous to help remedy a low energy jagged ultrasound signal, as shown in Panel (b) of FIG. 5. The covariance matrix may be constructed by taking the Hilbert transformed RF ultrasound samples within a region of ultrasound data. The region can be a clear echo region. The region may also be consistent with the length of the pulse period of the ultrasound data. The covariance matrix can be analyzed by a Hilbert transform.

The analytic pulse echo signal that results from the Hilbert transform of a single pulse period, denoted by “k” for kth pulse period, may be expressed as:

$$H ( z , k ) = H ( m , k ) = R ( m , k ) exp ( j [ ω t + φ ( m , k ) ] )$$

where R is amplitude, ω=2πf is the carrier frequency, φ is signal phase, and z is depth and corresponds, if discretized, to the m^{th} a/d sample. The analytic pulse echo signal can be used to find elements of the covariance matrices. In some cases, the matrices can be N-by-N covariance matrices. In some cases, the covariance matrices can be from the same depth but two different pulse periods. These pulse periods can each be a known number of pulse periods from pulse period k. These pulse periods can be denoted by indices p and q. The covariance matrix summand can arise from a fixed depth, z or m. The covariance matrix can be an outer product matrix described by

$$K ( p , q , m ) = H ( m , p ) * {H}^{′} ( m , q )$$

where p={0 . . . N−1} and q={0 . . . N−1}, and the apostrophe can denote complex conjugation.

The completed covariance matrix can sum K(p, q, m) from a group of adjacent depths m={m0, m1, m2, . . . , mR}. Plugging in H from above into this covariance expression yields the following:

$$K ( p , q , m ) = R ( m , p ) ( j [ ω t + φ ( m , p ) ] \cdot R ( m , q ) exp ( - j [ ωt + φ ( m , q ) ] )$$

which is further simplified to:

$$K ( p , q , m ) = R ( m , p ) \cdot R ( m , q ) \cdot exp ( j [ φ ( m , p ) - φ ( m , q ) ] .$$

The covariance matrix arising from contributions across a series of contiguous depths spanning the specific tympanic membrane echo can be denoted by:

$$K ( p , q ) = {\sum }_{m = m 0}^{mR} R ( p , m ) \cdot R ( q , m ) \cdot exp ( j [ φ ( m , p ) - φ ( m , q ) ] )$$

which is written more simply as

$$K ( p , q ) = {\sum }_{m = m 0}^{mR} {R}_{m} ( p ) {R}_{m} ( q ) {e}^{j Δ}$$

where

$$Δ = Δ ( p , q ) = {φ}_{m} ( p ) - {φ}_{m} ( q ) .$$

and Δ can be a difference in phase sampled at one depth, m, at two different times, p and q. This can also be known as the time rate of change in phase of an ultrasound signal.

This can avoid demodulation because the carrier frequency at the start of the description of K is effectively subtracted out by multiplication of one exponential by a second which has been conjugated.

The covariance matrix may be calculated based on the above mathematical description. With the resulting matrix, the eigenvectors and eigenvalues may be denoted by:

$$K \vert {v}_{1} , {v}_{2} , {v}_{3} … {v}_{N} \vert = \vert \begin{matrix}{λ}_{1} & 0 & & 0 \\ 0 & {λ}_{2} & & \\ & & ⋱ & 0 \\ 0 & & 0 & {λ}_{N}\end{matrix} \vert \vert {v}_{1} , {v}_{2} , {v}_{3} … {v}_{N} \vert .$$

where v₁, v₂, v₃ . . . v_{N} is the set of eigenvectors and (λ₁, λ₂, λ₃ . . . ) are the eigenvalues

In some cases, the displacement vectors for the plurality of related covariance matrices are calculated based on a target quality signal ratio. The target quality signal ratio can be a value between 0.00 and 1.00. In some cases, the target quality signal ratio can be 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.00.

In some cases, the systems can further comprise a processor configured to stitch together segments of the set of displacement vectors to generate a time trace of the position of the tissue. In some cases, the time trace can be the first trace. In some cases, the time trace can be the second trace. In some cases, the time trace can be the third trace.

In some cases, systems of determining a time-dependent position of a tissue in response to a stimulus can further comprise a processor configured to analyze one or more eigenvectors of the covariance matrix. In some cases, the one or more eigenvectors can each comprise an orbital rotation associated with a phase of the ultrasound data. It is noted that there are many ways to express the eigenvectors and eigenvalues for a covariance operator. In some cases, the membrane motion may be generally contained within the first eigenvector. In some cases, the membrane motion may be generally contained within the second eigenvector. In some cases, the membrane motion may be generally contained within the third eigenvector. In some cases, the membrane motion may be generally contained within a different eigenvector.

In some cases, eigenvectors can have similarity of phase track behavior across many depths, as shown in Panels (a), (b), and (c) of FIG. 5. In some cases, the overall signal energy may be clustered in just a small number of eigenvectors. In some cases, the eigenvectors {v₁, v₂, v₃ . . . v_{N}} can constitute an orthonormal basis set. In some cases, the ratio of each eigenvalue to the sum of all eigenvalues can be the fraction of signal class energy represented in an associated eigenvector. In some cases, the eigenvalue energy can be contained within one eigenvector. In some cases, places where the signal is energy depleted such as in Panel (b) of FIG. 5 may show a concurrent drop in the energy fraction contained in the first eigenvector. In some cases, to quantify this, a target signal quality ratio may be expressed by:

$$M = {λ}_{1} / {\sum }_{m = 1}^{N} {λ}_{m} .$$

M can be evaluated at various depths along the ultrasound beam over the number of pulse periods contained in the covariance calculation. When there is no target present, M can be a value between 0 and 1. In some cases, M can have higher variance than when a target is present. These facets of the quality ratio behavior are seen, for example, in FIG. 10.

A physical model of tympanic membrane movement can show continuous circulation of phase without jumps or discontinuities. Panels (a), (b), and (c) of FIG. 6 illustrate an example of a non-physical phase behavior that may occur with tracking the tympanic membrane together with an example of unpacking and correcting it.

Panel (a) of FIG. 6 shows an example of an eigenvector of the covariance from Region 2 of FIG. 4. In some cases, an eigenvector can show, for example, an amplitude drop. The observed drop may occur because the reflection strength from the tympanic membrane becomes low relative to the reflection from nearby tissue (such as the umbo) which is stationary (nonmoving) by comparison. Panel (a) of FIG. 6 shows an example of one of the orbital cycles of the path, which can be biased. Panel (b) of FIG. 6 shows the same eigenvector as in Panel (a) of FIG. 6, but includes, for example, a pre-inserted bias so that all orbits of the signal are about the complex plane origin. Panel (c) of FIG. 6 shows, for example, the cumulative phase change of the path in Panel (a) of FIG. 6 as trace 1, and the cumulative phase change of the path in Panel (b) of FIG. 6 as trace 2. The difference between traces 2 and 1 can be seen, for example in the dashed trace 3. There can be, for example a 2π difference due to the bias correction from Panel (a) of FIG. 6 to Panel (b) of FIG. 6.

In some cases, systems of determining a time-dependent position of a tissue in response to a stimulus can further comprise a processor configured to apply a bias to each of the one or more eigenvectors such that the eigenvector circles an origin in phase space. While adding a corrective bias to the data, such as is shown Panel (b) of FIG. 6, may be one solution to the problem of non-physical phase extractions, it may add its own artifact to the data.

FIG. 7 shows, for example, that the ultrasound signal can be a linear sum of phasors in the complex plane. As shown, motion associated with a particular phasor may be obscured if the phasor orbital center is biased away from the origin of the complex plane. Panel (a) of FIG. 7 illustrates phasor 2, which can be the signal of interest, which can orbit away from the origin because of a bias from Phasor 1. Panel (b) of FIG. 7 illustrates that phasor 2, which can be the signal of interest, can orbit around the complex plane origin. Panel (c) of FIG. 7 illustrates an example of the two phasor behavior of Panel (a) of FIG. 7 with ultrasound signal data from brain tissue. In some cases, phasor 1 can be the slow-moving phasor, and a particulate in blood flow in the middle cerebral artery is phasor 2, which can be the fast-moving phasor. Panel (d) of FIG. 7 illustrates the example of Panel (b) of FIG. 7 with same brain/particulate data, but the SLOW brain phasor can be filtered out with a high-pass filter (clutter filter), leaving just the embolus signal as the residual phasor orbiting the complex origin. Panel (e) of FIG. 7 shows an exemplary calculation of embolus position based on data with Panel (a) of FIG. 7 type-bias, which can show inaccurately very little motion of the embolus moving in blood flow. Panel (f) of FIG. 7 can show, for example, a calculation of embolus position based on data with the character of Panel (b) of FIG. 7 and Panel (d) of FIG. 7, which can result in accuracy improvements in calculating the motion of the embolus.

As shown in Panel (e) of FIG. 5, in some examples, there can be a dip in the signal to noise in the region near the target. The target can be, for example, the tympanic membrane. Covariance analysis across the entire same region containing the target may, in some cases, diminish broadband noise and make the motion pathway and dynamics more apparent. In some cases, noise process covariance (data with no target present) results in the use of more eigenvectors to describe the process than for target covariance (data with a target present). Covariance analysis with a target present can, in some cases, result in 1 or single digit components to describe target dynamics. The principal eigenvector (with the largest eigenvalue) can, in some cases, describe target motion which has contribution from all the a/d samples that are within the target echo. In some cases, the broadband noise may tend to be separately analyzable from the signal.

In some cases, systems of determining a time-dependent position of a tissue in response to a stimulus can further comprise a processor configured to use a resulting biased eigenvector to calculate a second trace of the position of the tissues. In some cases, analyzing the one or more eigenvectors can further comprise analyzing membrane motion information of the one or more eigenvectors. In some cases, analyzing the one or more eigenvectors can further comprise repeating the analysis in 10 millisecond (ms) steps. In some cases, the 10 ms steps can be in an adjustable 20 ms analysis window. In some cases, analyzing the one or more eigenvectors can further comprise analyzing depth range values. In some cases, analyzing the one or more eigenvectors can further comprise creating a set of principal eigenvectors at each 10 millisecond (ms) step. In some cases, analyzing the one or more eigenvectors can further comprise creating a set of principal eigenvectors within each 20 ms analysis window.

In some examples, repeating the calculation of the principal eigenvector of the covariance analysis across the depth extent and the time extent of the signal can be shown in Panel (A) of FIG. 3. In the example shown in Panel (A) of FIG. 3, in some cases, the data stream can extend for 1 second duration during the tympanic membrane data capture. This can be, for example, about 5000 pulse periods of data over which to do a covariance analysis. In some cases, the analysis may be repeated in 10 ms steps. In some cases, the analysis may be further repeated over the 20 ms window from the early edge of the signal to the latest edge of the signal. In some cases, the analysis can be further repeated over the M₁ values central to the tympanic membrane silhouette. For example, the top 80 M₁ values at each time position in FIG. 10 relative to the silhouette of Panel (A) of FIG. 3 and Panel (B) of FIG. 3. In some cases, the stimulus can be an excitation. In some cases, the excitation can be a physical, electrical, or electromagnetic excitation. In some cases, the stimulus can be a pneumatic excitation. In some cases, the pneumatic excitation can be an air puff.

For example, the set of principal eigenvectors that can result, 1 per window position, at each of 100 windows in 10 ms increments across the 1 second time range, may be represented as

$$V = \{ {v}_{1}^{( 1 )} , {v}_{1}^{( 2 )} , {v}_{1}^{( 3 )} , … , {v}_{1}^{( N )} \} .$$

The membrane motion information contained in an eigenvector may obtained by the following expression

$${\vec{d}}_{k} = \frac{U ( ∠ {v}_{1}^{( k )} ) λ}{4 π}$$

where U is the “phase unwrap operator”. This expression can be the unwrapped eigenvector phase, factored by one wavelength of carrier frequency for every 4π radians of phase change. \vec{d} can be a vector which has one value for every pulse period included in the covariance analysis window.

Similar to the set of eigenvectors above, there is a set of displacement vectors can be denoted by

$$D = \{ {\vec{d}}_{1} , {\vec{d}}_{2} , {\vec{d}}_{3} , … , {\vec{d}}_{N} \} .$$

Panel (a) of FIG. 8 shows an example of the set D of vectors containing positional change during each covariance calculation time interval. These individual segments can show the relative position change made by the tympanic membrane. In some cases, they may be “stitched” to their adjacent neighbors. Panel (b) of FIG. 8 shows, for example, these curves with magnification and presents the fit they have with each other in their overlapping regions. Panel (c) of FIG. 8 shows an example of the segments stitched together end-to-end and revealing the tympanic motion.

FIG. 9 illustrates the phase profile plotted, for example, on the backdrop of the original RF ultrasound data.

FIG. 10 illustrates an example of quality Metric M, calculated across 20 ms wide windows which are spaced in 10 ms increments for a total of 1 second duration. The noise background can be, for example, close to a level of 0.2, compared to the “target” which is close to 1.0 with occasional dips across the time span and across a depth span equal to the ultrasound sample volume size. The path at the top can be the position of the maxim value of M across depth, at each 10 ms increment.

### Methods of Autoregression

It may be beneficial to further correct the unwrapped phase. For example, near a pressure transition the phase may exhibit singularities, e.g., jumps in the phase that would suggest unphysically fast motion of the tympanic membrane. Such a singularity is referred to as an inaccuracy or “alias” in the resulting motion detection. This alias in many cases is wrong. Optionally, antialiasing methods and systems described below may be used to address these non-physical movements.

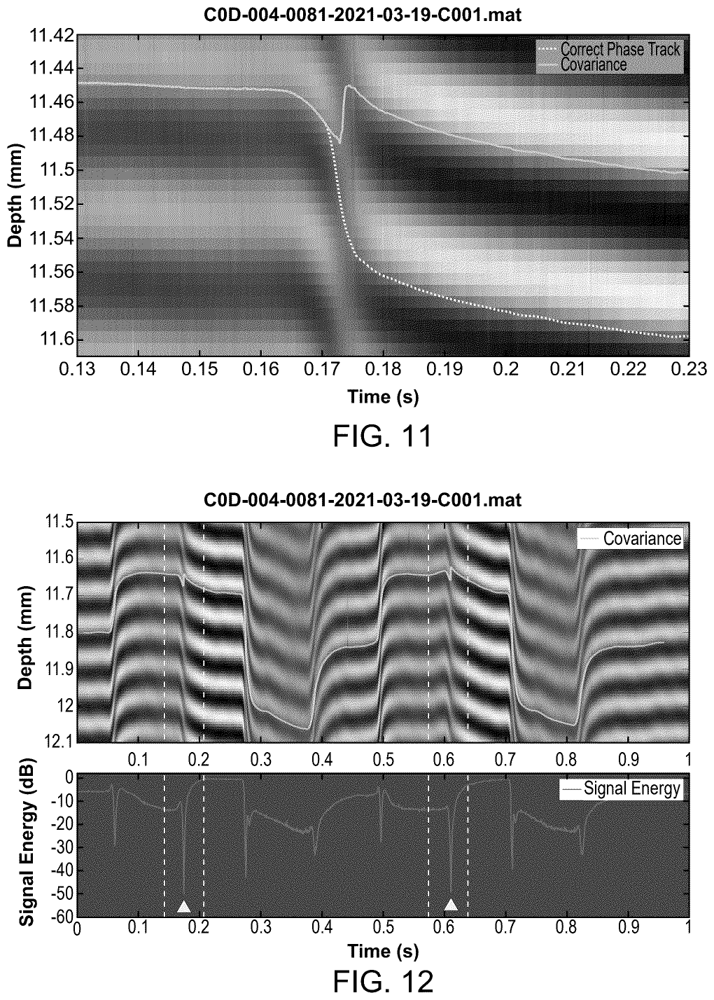

FIG. 11 shows a zoomed in example of the covariance trace following an RF phase track of the TM similar to FIG. 9.

FIG. 12 shows that, for example, the covariance trace remains faithful to the phase track RF data except in two places (Time≈0.18 s and Time≈0.62). These two covariance misses occur during pressure transition regions, and they are the two places where the signal energy is the lowest (indicated by triangles). As shown in FIG. 12, the covariance may “hop” from one phase track to another, jumping away from the correct path. This may occur during pressure transition regions when the signal energy is at its lowest.

Processing of ultrasound data may comprise analyzing the frequency content of a complex demodulation of the RF echo across pulse periods. The frequency of the slow time ultrasound shift may be proportional to the velocity, so frequency estimation techniques may be applied across slow time to track the velocity of a reflector over time. Autoregression (AR) models are a technique that can be used to estimate the frequency spectrum of a signal.

In some cases, methods of determining a time-dependent position of a tissue in response to a stimulus can further comprise analyzing a frequency content of the ultrasound data across the plurality of pulse periods. In some cases, the methods can further comprise determining one or more regions where the first trace comprises a non-physical tissue position or movement. In some cases, methods of determining a time-dependent position of a tissue in response to a stimulus can further comprise analyzing a frequency content of the ultrasound data across the plurality of pulse periods. In some cases, the methods can further comprise substituting at least a portion of the second trace for the first trace in the one or more regions. In some cases, determining one or more regions where the first trace can comprise a non-physical tissue position or movement can comprise utilizing autoregression. In some cases, the autoregression can be a third order autoregression. In some cases, the autoregression can be higher than a third order autoregression. In some cases, the autoregression can be a first order autoregression. In some cases, the autoregression can be a second order autoregression. In some cases, the autoregression can be a fourth order autoregression. In some cases, the autoregression can be a fifth order autoregression. In some cases, the autoregression can be more than a fifth order autoregression.

Methods of improving accuracy of a time-dependent position determination of a tissue in response to a stimulus disclosed herein may comprise generating a frequency spectrum of a signal trace. Methods can further comprise applying autoregression to generate one or more poles. The number of poles generated can be one pole, two poles, three poles, or more than three poles. The method can further comprise eliminating or selecting a pole of the one or more poles using a feature of the one or more poles. The number of poles that can be eliminated or selected are one pole, two poles, three poles, or more than three poles. The method can further comprise generating an improved signal trace of a position of the tissue from remaining or selected poles of the one or more poles.

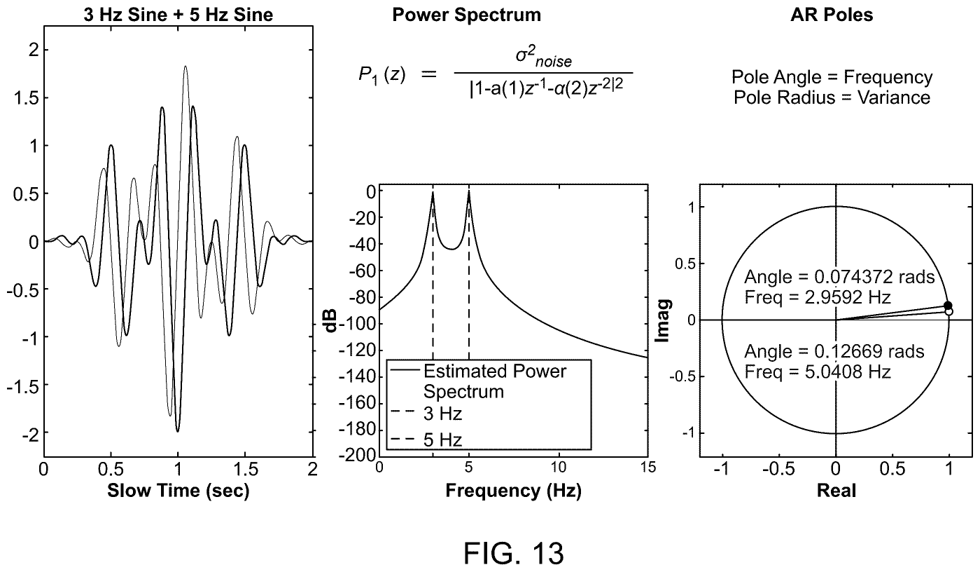

FIG. 13 illustrates an example of a second order AR model to estimate the frequency spectrum of a signal. The left panel shows an artificial signal created by summing a 3 Hz sinusoid and a 5 Hz sinusoid. The middle panel shows the frequency spectrum of a signal, which estimated using the equation shown. The middle panel shows the estimated frequency spectrum with peaks at 3 Hz and 5 Hz as expected. The coefficients a(1) and a(2) in the equations are the AR coefficients which were solved for using the Burg algorithm. The right panel shows that the poles of the equation in the middle contain information about the frequency estimate. The angle of each pole on the complex unit circle can correspond to a peak in the frequency spectrum, with +/−π equal to +/− Nyquist Frequency. The radius, r, of each pole informs the variance or width of the frequency spectrum and can be proportional to 1−r.

AR models may be implemented using the methods of Young Bok Ahn and Song Bai Park, “Estimation of mean frequency and variance of ultrasonic Doppler signal by using second-order autoregression,” in IEEE Transactions on Ultrasonics, Ferroelectrics, and Frequency Control, vol. 38, no. 3, pp. 172-182, May 1991, doi: 10.1109/58.79600, which is incorporated herein by reference for all purposes.

In some examples, the frequency content of a signal can be estimated using an AR model. The order of the AR model may determine how many different frequency peaks the AR model can separate. In FIG. 13, for example, the input signal has two frequencies that were to be separated, so a second order AR model returned two frequency peaks.

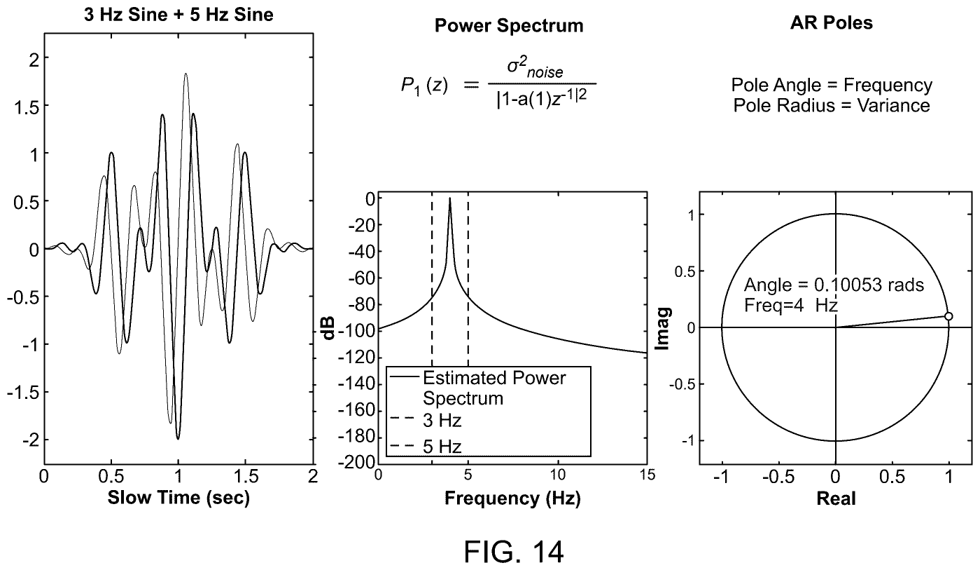

FIG. 14 shows an example of using a first order AR model to estimate the frequency spectrum of a signal. A first order AR model can return one frequency peak in the spectral estimate, so if the input signal contains two frequencies, the AR model can return the average of those two frequencies.

AR models may be implemented using the methods of C. Kasai, K. Namekawa, A. Koyano and R. Omoto, “Real-Time Two-Dimensional Blood Flow Imaging Using an Autocorrelation Technique,” in IEEE Transactions on Sonics and Ultrasonics, vol. 32, no. 3, pp. 458-464, May 1985, doi: 10.1109/T-SU.1985.31615, which is incorporated herein by reference for all purposes.

FIG. 13 and FIG. 14 show, for example, the application of AR frequency estimation to a simple artificial signal, but this technique may be applied to a slow time ultrasound signal to estimate the velocity of the TM.

The AR processing techniques described herein are not limited to first or second order model. In some cases, the autoregression can be applied to one or more points of the signal trace. In some cases, the autoregression can be applied to two points of the signal trace, three points of the signal trace, four points of the signal trace, five points of the signal trace, or more than five points of the signal trace. In some cases, the autoregression can be a third order autoregression. In some cases, the autoregression can be higher than a third order autoregression. In some cases, the order of the autoregression can correspond with the number of frequency peaks of the signal trace that are separated. In some cases, the signal trace can comprise a covariance motion detection trace. In some cases, the autoregression can be applied to one or more eigenvectors of the covariance motion detection trace. In some cases, the autoregression can be applied to two eigenvectors, three eigenvectors, four eigenvectors, five eigenvectors, or more than five eigenvectors of the covariance motion detection trace. In some cases, the methods of improving accuracy of a time-dependent position determination of a tissue in response to a stimulus can further comprise estimating a mean frequency of the signal trace. In some cases, estimating a mean frequency of the signal trace can comprise using an angle value between each of the one or more poles and a center of a complex unit circle of a complex plane. In some cases, the angle value can be calculated by taking an inverse tangent over the imaginary component of the pole of the real component of the pole.

In some cases, the methods of improving accuracy of a time-dependent position determination of a tissue in response to a stimulus can further comprise estimating variance of the frequency of the signal trace. The variance can be estimated using a radius value between each of the one or more poles and a center of a complex unit circle of a complex plane. In some cases, the methods of improving accuracy of a time-dependent position determination of a tissue in response to a stimulus can further comprise estimating a velocity. In some cases, the velocity can be represented by the signal trace using a radius value between each of the one or more poles and a center of a complex unit circle of a complex plane. In some cases, the number of poles can be one pole, two poles, three poles, four poles, five poles, or more than five poles.

In some cases, the methods of improving accuracy of a time-dependent position determination of a tissue in response to a stimulus can further comprise plotting a velocity trace over time of the signal trace. In some cases, the velocity trace can be plotted over time of the signal trace by plotting the angle between the one or more poles and a center of a complex unit circle of a complex plane.

The radius of each pole can be used to determine the variance of the estimated frequency spectrum using, for example, the equation below where T is the pulse period and r is the pole radius.

$$f = \frac{1}{2 π T} {tan}^{- 1} ( \frac{Im ( Pole )}{Re ( Pole )} ) v = 72 \text{.54} ( \frac{\frac{mm}{s}}{radian} ) * angle ( Pole )$$

$${σ}^{2} = \frac{2}{{T}^{2}} ( 1 - r )$$

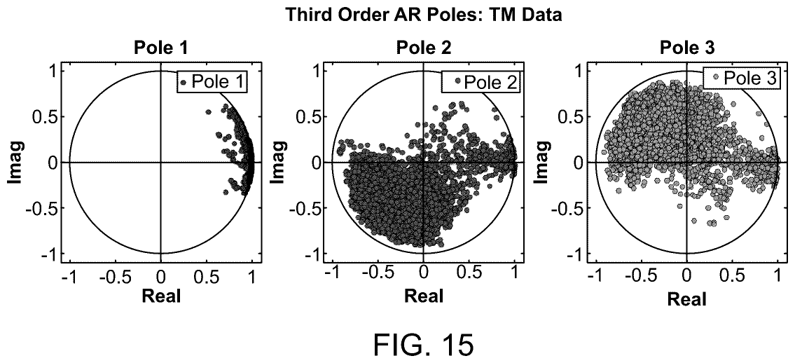

FIG. 15 shows, for example, the poles of the spectral estimate that result when a 12 point, third order AR model is applied to the first eigenvector of the covariance. Pole 1 can primarily estimate the lower frequency velocity of the TM whereas poles 2 and 3 can primarily estimate velocities from high frequency noise.

The poles shown in FIG. 15 can be found by solving for the roots of the denominator of a third order estimate for the power spectral density.

$${P}_{3} ( z ) = \frac{{σ}_{noise}^{2}}{{\vert 1 - a ( 1 ) {z}^{- 1} - a ( 2 ) {z}^{- 2} - a ( 3 ) {z}^{- 3} \vert}^{2}}$$

To determine the roots of the denominator (“poles”), a cubic formula, shown below, may be used to find the zeros of the third order polynomial in the denominator of the equation above. These zeros of the cubic polynomial are the poles of the third order AR model, and these three poles provide the frequency estimation used for ultrasound data processing.

$$A = \frac{\sqrt[3]{- 2 {p}^{3} + 9 pq - 27 r + 3 \sqrt{3} \sqrt{- {p}^{2} {q}^{2} + 4 {q}^{3} + 4 {p}^{3} r - 18 pqr + 27 {r}^{2}}}}{3 \sqrt[3]{2}}$$

$$B = \frac{- {p}^{2} + 3 q}{9 A}$$

$$Root 1 : {a}_{1} = - \frac{p}{3} + A - B$$

$$Root 2 : {a}_{2} = - \frac{p}{3} + ( \frac{- 1 - i \sqrt{3}}{2} ) A - B ( \frac{- 1 + i \sqrt{3}}{2} )$$

$$Root 3 : {a}_{3} = - \frac{p}{3} + ( \frac{- 1 + i \sqrt{3}}{2} ) A - B ( \frac{- 1 - i \sqrt{3}}{2} )$$

Pole 1 of the third order AR model corresponds to root 1 of the above cubic formula. Poles 2 and 3 are then “angular conjugates” of pole 1 just as roots 2 and 3 are conjugate combinations of root 1.

As shown in FIG. 15, applying a 12 point third order AR model to the first eigenvector of the covariance can yield three poles.

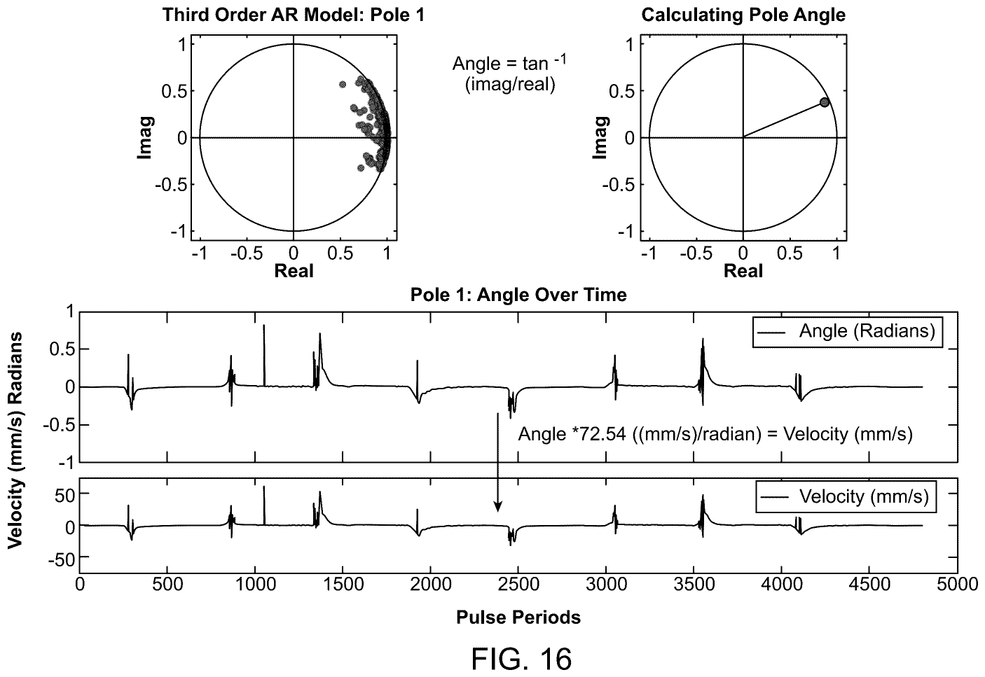

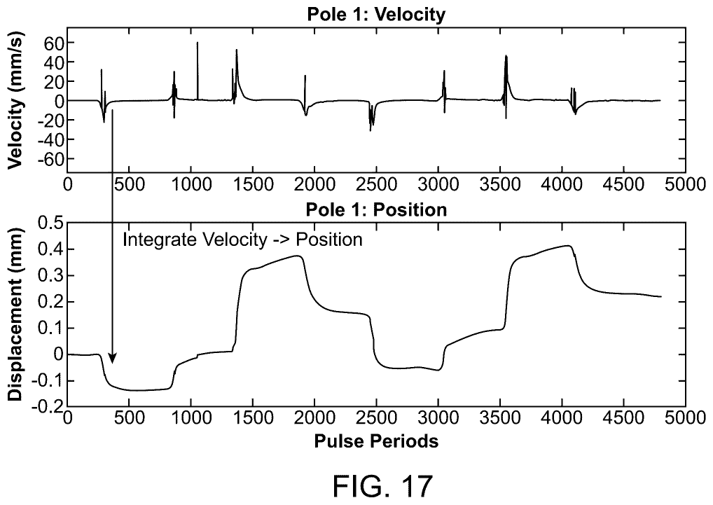

As shown in FIG. 16, velocity over time can be determined by plotting the angle of a given pole for all pulse periods. This angle can be converted to velocity using the equations shown above. If the velocities estimated by pole 1 are integrated over time to get displacement (FIG. 17), the integral can be compared to the pole 1 position to trace to the covariance position trace.

In some cases, the methods of improving accuracy of a time-dependent position determination of a tissue in response to a stimulus can further comprise calculating one or more displacement values between a position of the velocity trace and a position of the signal trace. In some cases, the determination of the one or more displacement values can comprise integrating the velocities of the velocity trace. In some cases, the features of the one or more poles can further comprise the radius, the angle, the velocity, complex plane distance between the one or more poles, change in velocity between the one or more poles, change in inertial velocity between the one or more of poles, or momentum of the one or more poles, or any combination thereof. In some cases, the one or more poles can be segmented into one or more frequency groups based on frequency. In some cases, the one or more poles can be segmented into two frequency groups. In some cases, the one or more poles can be segmented into three frequency groups. In some cases, the one or more poles can be segmented into more than three frequency groups.

Table 1 below shows an exemplary summary of the 1^{st}, 2^{nd} and 3^{rd} order autoregression formulas for calculating model parameters (poles) from ultrasound shift sequence data {z₁, z₂, z₃, . . . , z_{N}}.

| AUTOREGRESSION | 1^{st} order | 2^{nd} order | 3^{rd} order |
|---|---|---|---|
| Parameters to calculate | a₁ | a₁, a₂ | a₁, a₂, a₃ |
| Link between Doppler sequence time series data {z₁, z₂, z₃, ... , z_{N}} and poles | ${a}_{1} = \frac{1}{N - 1} {\sum }_{m = 1}^{N - 1} {z}_{m} {z}_{m + 1}^{*}$ | ${d}_{1} = {\sum }_{m = 2}^{N} {\vert z ( m ) \vert}^{2} + {\vert z ( m - 1 ) \vert}^{2}$ | See in text above |
| | | ${k}_{1} = \frac{- 2 {\sum }_{n = 2}^{N} z ( n ) {z}^{*} ( n - 1 )}{{d}_{1}}$ | |
| | | e^{f}(n) = z(n) + k₁z(n − 1) | |
| | | ${e}^{b} ( n ) = z ( n - 1 ) + {k}_{1}^{*} z ( n )$ | |
| | | d₂ = d₁ (1 − \|k₁\|) − \|e^{f}(2)\| − \|e^{b}(N)\| | |
| | | ${k}_{2} = \frac{- 2 {\sum }_{n = 3}^{N} {e}^{f} ( n ) {e}^{b} ( n - 1 )}{{d}_{2}}$ | |
| | | a₁ = k₂ | |
| | | a₂ = k₁ + k₂k₁^{*} | |
| Velocity associated with model parameter | a₁/T | ${\overline{ω}}_{1} = ∠ \frac{{a}_{1}}{T}$ | ${\overline{ω}}_{1} = ∠ \frac{{a}_{1}}{T}$ |
| | | ${\overline{ω}}_{2} = ∠ \frac{{a}_{2}}{T}$ | ${\overline{ω}}_{2} = ∠ \frac{{a}_{2}}{T}$ |
| | | | ${\overline{ω}}_{3} = ∠ \frac{{a}_{3}}{T}$ |
| Signal spectral width (variance) | ${σ}_{1}^{2} = \frac{2}{{T}^{2}} [ 1 - \vert {a}_{1} \vert ]$ | ${σ}_{1}^{2} = \frac{2}{{T}^{2}} [ 1 - \vert {a}_{1} \vert ]$ | ${σ}_{1}^{2} = \frac{2}{{T}^{2}} [ 1 - \vert {a}_{1} \vert ]$ |
| | | ${σ}_{2}^{2} = \frac{2}{{T}^{2}} [ 1 - \vert {a}_{2} \vert ]$ | ${σ}_{2}^{2} = \frac{2}{{T}^{2}} [ 1 - \vert {a}_{2} \vert ]$ |
| | | | ${σ}_{3}^{2} = \frac{2}{{T}^{2}} [ 1 - \vert {a}_{3} \vert ]$ |

FIG. 16 shows an exemplary plot of the pole angle over time both in velocity and radians. Pole angle can be calculated by taking the inverse tangent over the imaginary component of a given pole over the real component of a given pole. This angle can be plotted over time and then converted to velocity using the equations shown above.

FIG. 17 shows a plot of the pole velocity and position for all pulse periods. Once the velocity is calculated for all pulse periods, it can be integrated over time to get the displacement of the TM.

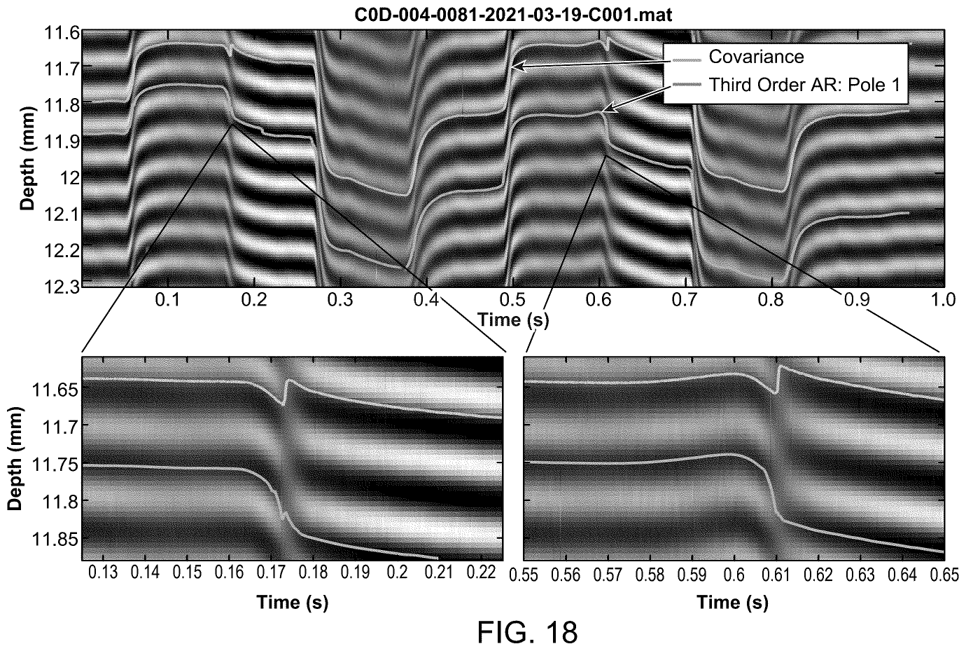

FIG. 18 shows a comparison of the covariance method with pole 1 of the third order AR model.

In some cases, generating the optimized signal trace can further comprise replacing the one or more poles of the signal trace with one or more poles of a different frequency group. In some cases, the one or more poles of the signal trace can be replaced when the absolute error between one or more of the features of the one or more poles are above a threshold value. In some embodiments, the threshold value is a value calculated by one or more of the pole selection algorithms of Table 3. In some embodiments, the poles are of opposite signs. In some embodiments, the threshold value is between 0.1 and 5.0. In some cases, the threshold value is about 0.5, 1.0, 1.5, 2.0, 2.5, 3.0, 3.5, 4.0, 4.5, 5.0, or about more than 5.0. In some cases, the one or more poles of the signal trace can be replaced when the absolute error between one or more of the features of the one or more poles are below a threshold value. In some embodiments, the threshold value is a value calculated by one or more of the pole selection algorithms of Table 3. In some embodiments, the poles are of opposite signs. In some embodiments, the threshold value is between 0.1 and 5.0. In some cases, the threshold value is about 0.5, 1.0, 1.5, 2.0, 2.5, 3.0, 3.5, 4.0, 4.5, 5.0, or about more than 5.0. In some cases, the threshold value can be π/3 and the poles can be of opposite signs. In some cases, the threshold value can be π/2, π/3, π/4, π/5, π/6, π/7, π/8, π/9, π/10, or less than π/10. In some cases, the poles can be of the same sign.

### System for Monitoring Tissue Movement in Response to a Stimulus

In some cases, disclosed herein can be a system comprising a processor configured to apply autoregression to one or more points of the signal trace. In some cases, the autoregression can be applied to two points of the signal trace, three points of the signal trace, four points of the signal trace, five points of the signal trace, or more than five points of the signal trace. In some cases, the autoregression can be a third order autoregression. In some cases, the autoregression can be higher than a third order autoregression. In some cases, the order of the autoregression can correspond with the number of frequency peaks of the signal trace that are separated. In some cases, the signal trace can comprise a covariance motion detection trace. In some cases, the autoregression can be applied to one or more eigenvectors of the covariance motion detection trace. In some cases, the autoregression can be applied to two eigenvectors, three eigenvectors, four eigenvectors, five eigenvectors, or more than five eigenvectors of the covariance motion detection trace. In some cases, the systems comprising a processor configured to improve accuracy of a time-dependent position determination of a tissue in response to a stimulus can further comprise estimating a mean frequency of the signal trace. In some cases, estimating a mean frequency of the signal trace can comprise using an angle value between each of the one or more poles and a center of a complex unit circle of a complex plane. In some cases, the angle value can be calculated by taking an inverse tangent over the imaginary component of the pole of the real component of the pole.

In some cases, the systems comprising a processor configured to improve accuracy of a time-dependent position determination of a tissue in response to a stimulus can further comprise a processor configured to estimate variance of the frequency of the signal trace. The variance can be estimated by the processor using a radius value between each of the one or more poles and a center of a complex unit circle of a complex plane. In some cases, the systems comprising a processor configured to improve accuracy of a time-dependent position determination of a tissue in response to a stimulus can further comprise the processor estimating a velocity. In some cases, the velocity can be represented by the signal trace using a radius value between each of the one or more poles and a center of a complex unit circle of a complex plane. In some cases, the number of poles can be one pole, two poles, three poles, four poles, five poles, or more than five poles.

In some cases, the systems comprising a processor configured to improve accuracy of a time-dependent position can comprise a processor configured to determine velocity of a tissue in response to a stimulus. Determination of velocity can further comprise plotting a velocity trace over time of the signal trace. In some cases, the velocity trace can be plotted over time of the signal trace by plotting the angle between the one or more poles and a center of a complex unit circle of a complex plane.

The radius of each pole can be used to determine the variance of the estimated frequency spectrum using, for example, the equation below where T is the pulse period and r is the pole radius.

$$f = \frac{1}{2 π T} {tan}^{- 1} ( \frac{Im ( Pole )}{Re ( Pole )} ) v = 72 \text{.54} ( \frac{\frac{mm}{s}}{radian} ) * angle ( Pole )$$

$${σ}^{2} = \frac{2}{{T}^{2}} ( 1 - r )$$

FIG. 15 shows, for example, the poles of the spectral estimate that result when a 12 point, third order AR model is applied to the first eigenvector of the covariance. Pole 1 can primarily estimate the lower frequency velocity of the TM whereas poles 2 and 3 can primarily estimate velocities from high frequency noise.

The poles shown in FIG. 15 can be found by solving for the roots of the denominator of a third order estimate for the power spectral density.

$${P}_{3} ( z ) = \frac{{σ}_{noise}^{2}}{{\vert 1 - a ( 1 ) {z}^{- 1} - a ( 2 ) {z}^{- 2} - a ( 3 ) {z}^{- 3} \vert}^{2}}$$

To determine the roots of the denominator (“poles”), a cubic formula, shown below, may be used to find the zeros of the third order polynomial in the denominator of the equation above. These zeros of the cubic polynomial are the poles of the third order AR model, and these three poles provide the frequency estimation used for ultrasound data processing.

$$A = \frac{\sqrt[3]{- 2 {p}^{3} + 9 pq - 27 r + 3 \sqrt{3} \sqrt{- {p}^{2} {q}^{2} + 4 {q}^{3} + 4 {p}^{3} r - 18 pqr + 27 {r}^{2}}}}{3 \sqrt[3]{2}}$$

$$B = \frac{- {p}^{2} + 3 q}{9 A}$$

$$Root 1 : {a}_{1} = - \frac{p}{3} + A - B$$

$$Root 2 : {a}_{2} = - \frac{p}{3} + ( \frac{- 1 - i \sqrt{3}}{2} ) A - B ( \frac{- 1 + i \sqrt{3}}{2} )$$

$$Root 3 : {a}_{3} = - \frac{p}{3} + ( \frac{- 1 + i \sqrt{3}}{2} ) A - B ( \frac{- 1 - i \sqrt{3}}{2} )$$

Pole 1 of the third order AR model corresponds to root 1 of the above cubic formula. Poles 2 and 3 are then “angular conjugates” of pole 1 just as roots 2 and 3 are conjugate combinations of root 1.

As shown in FIG. 15, applying a 12 point third order AR model to the first eigenvector of the covariance can yield three poles.

As shown in FIG. 16, velocity over time can be determined by plotting the angle of a given pole for all pulse periods. This angle can be converted to velocity using the equations shown above. If the velocities estimated by pole 1 are integrated over time to get displacement (FIG. 17), the integral can be compared to the pole 1 position to trace to the covariance position trace.

In some cases, the systems comprising a processor configured to improve accuracy of a time-dependent position can comprise a processor configured to calculate one or more displacement values between a position of the velocity trace and a position of the signal trace. In some cases, the determination of the one or more displacement values can comprise integrating the velocities of the velocity trace. In some cases, the features of the one or more poles can further comprise the radius, the angle, the velocity, complex plane distance between the one or more poles, change in velocity between the one or more poles, change in inertial velocity between the one or more of poles, or momentum of the one or more poles, or any combination thereof. In some cases, the one or more poles can be segmented into one or more frequency groups based on frequency. In some cases, the one or more poles can be segmented into two frequency groups. In some cases, the one or more poles can be segmented into three frequency groups. In some cases, the one or more poles can be segmented into more than three frequency groups.

In some cases, generating the optimized signal trace can further comprise replacing the one or more poles of the signal trace with one or more poles of a different frequency group. In some cases, the one or more poles of the signal trace can be replaced when the absolute error between one or more of the features of the one or more poles are above a threshold value. In some embodiments, the threshold value is a value calculated by one or more of the pole selection algorithms of Table 3. In some embodiments, the poles are of opposite signs. In some embodiments, the threshold value is between 0.1 and 5.0. In some cases, the threshold value is about 0.5, 1.0, 1.5, 2.0, 2.5, 3.0, 3.5, 4.0, 4.5, 5.0, or about more than 5.0. In some cases, the one or more poles of the signal trace can be replaced when the absolute error between one or more of the features of the one or more poles are below a threshold value. In some cases, the threshold value can be π/3 and the poles can be of opposite signs. In some cases, the threshold value can be π/2, π/3, π/4, π/5, π/6, π/7, π/8, π/9, π/10, or less than π/10. In some cases, the poles can be of the same sign.

### Hybrid Method and Systems

A hybrid approach that combines the faithfulness of covariance with the ability to reliably follow phase tracks during pressure transitions of the AR estimation may be advantageous. A hybrid method or system may combine both covariance and autoregression processing algorithms into one method.

In some cases, an AR trace may be used to adjust the covariance. This adjustment may be applied to regions where the covariance fails to follow a “physical” phase track, i.e., one that conforms to physical understanding of membrane movement.

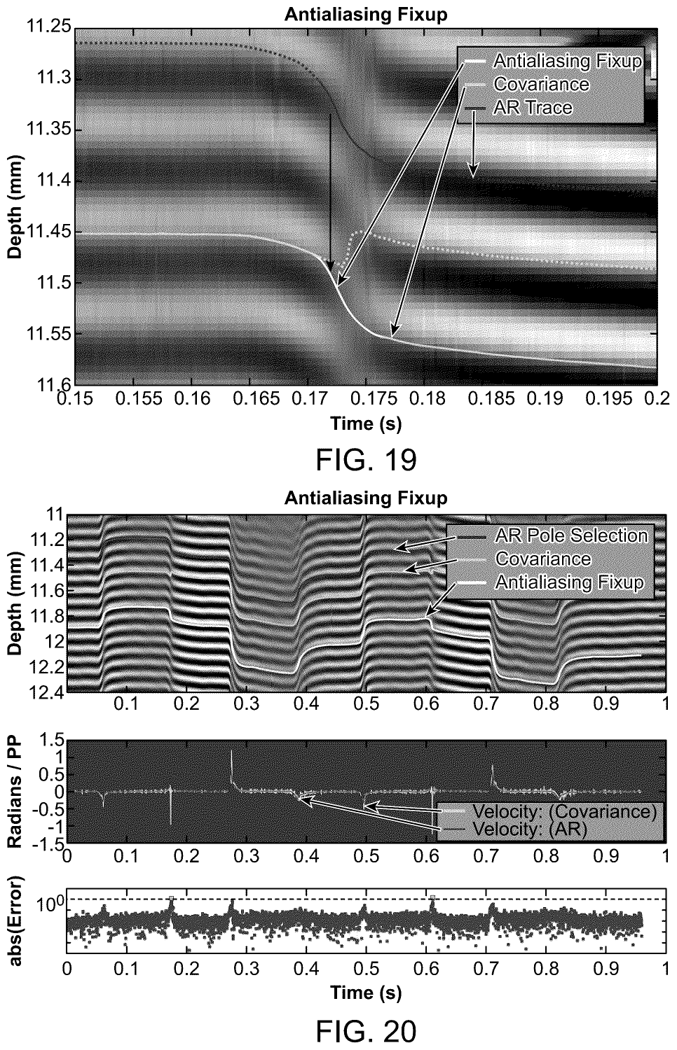

FIG. 19 provides an exemplary visual outline of the process of adjusting the covariance trace based on an AR analysis. As shown, the covariance trace may transition from one phase track to another. The region where the covariance misses the pressure transition can be a missed region. The missed region may be replaced with a section of the AR trace to create the antialiasing trace. This antialiasing trace may represent a combination of the covariance and AR processing.

FIG. 20 shows an example implementation of the antialiasing (hybrid) algorithm. Three different TM displacement traces are shown (top). The third order AR trace and the covariance trace may be combined to form the antialiasing trace. The velocity traces for the AR processing and the covariance are shown in the middle panel. The absolute error between the AR and covariance velocities is shown, for example in the bottom panel. When the difference between the absolute errors of the AR trace and the covariance trace is greater than π/3, and the velocities are opposite signs, the covariance displacement trace can be fixed up with the AR trace.

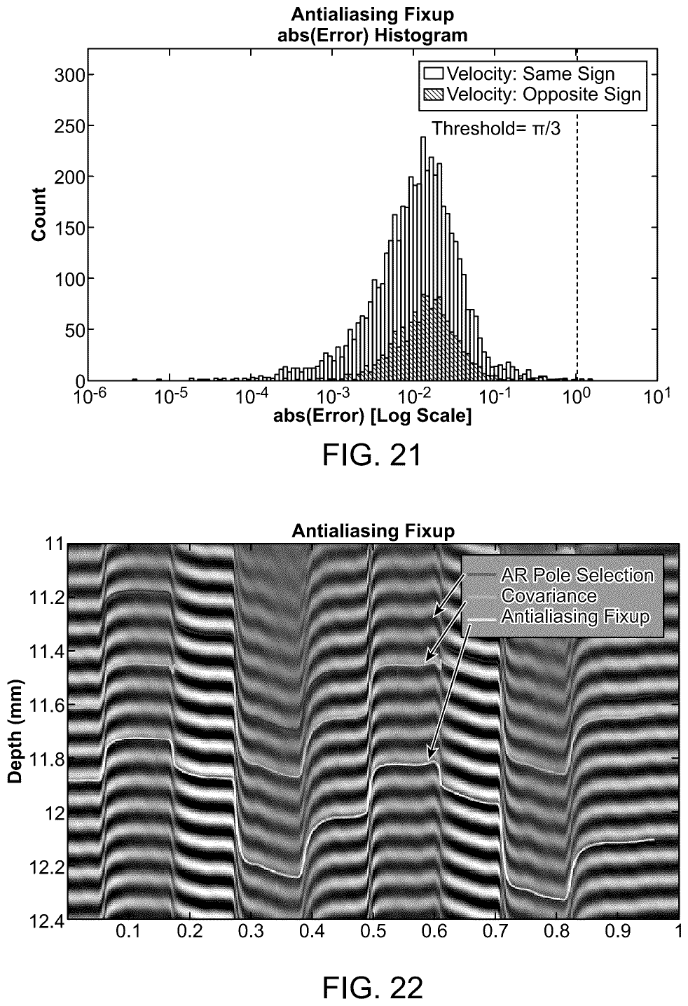

FIG. 21 shows an exemplary histogram of the absolute values of the error between the AR and covariance velocities.

FIG. 22 shows an exemplary result of the antialiasing procedure. The AR trace and covariance trace can be combined to form the antialiasing trace. This hybrid trace can be faithful to the RF phase track data and may not miss pressure transitions.

The method above may be performed in close temporal proximity to the covariance calculation or as a post processing routine.

Systems for improving a time-dependent position determination are disclosed herein. The systems can comprise a covariance analysis model. The covariance analysis model can comprise executable instructions which when executed by a processor can be configured to derive one or more covariance matrices from ultrasound data. The processor can be configured to derive one, two, three, four, five, or more than five covariance matrices form ultrasound data. The processor can be further configured to calculate one or more eigenvectors of the one or more covariance matrices. The processor can be configured to calculate one, two, three, four, five, or more than five eigenvectors of the one or more covariance matrices. The processor can be further configured to generate a trace from the one or more eigenvectors. The processor can be configured to generate a trace from one, two, three, four, five, or more than five eigenvectors. The system can further comprise one or more autoregressions configured to generate one or more poles. The autoregressions can be configured to generate one pole, two poles, three poles, or more than three poles. The system can further comprise an antialiasing model comprising executable instructions which when executed by a processor can be configured to select one or more areas of the trace that deviates from a phase track. The processor can be configured to select one, two, three, four, five, or more than five areas of the trace that deviate from a phase track. The system can further comprise a signal trace model comprising executable instructions which when executed by a processor can be configured to eliminate one or more poles of the one or more areas of the trace selected by the antialiasing model. The signal trace model can be configured to eliminate the one or more poles based at least in part on an effect of one or more features of the one or more poles. The signal trace model can be further configured to select one or more replacement poles of one or more alternative signal traces to replace the one or more poles eliminated. In some cases, the signal trace model can be configured to select the one or more replacement poles based at least in part on an effect of one or more features of the one or more replacement poles. In some cases, the signal trace model can be configured to select one, two, three, or more than three replacement poles. In some cases, the system can further comprise an output generator configured to generate an improved signal trace of a position of the tissue from the one or more replacement poles output by the antialiasing model.

In some cases, the one or more features can comprise radius of the one or more poles to a center of a complex unit circle, angle between the one or more poles and the center of a complex unit circle, velocity of the angle between the one or more poles, complex plane distance between the one or more poles, change in velocity between the one or more poles, change in inertial velocity between the one or more of poles, or momentum of the one or more poles, or any combination thereof.

### Pole Selection

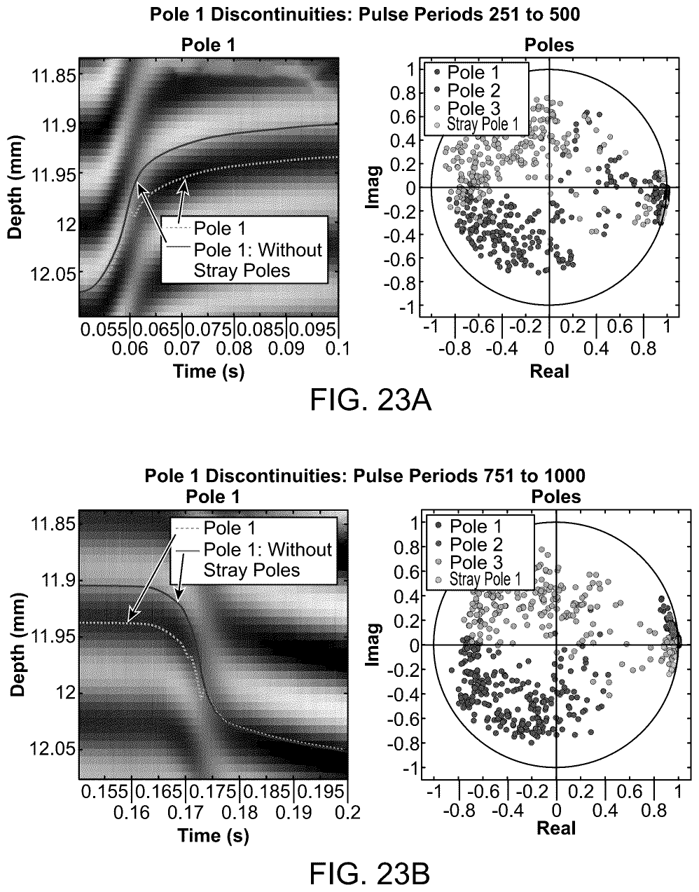

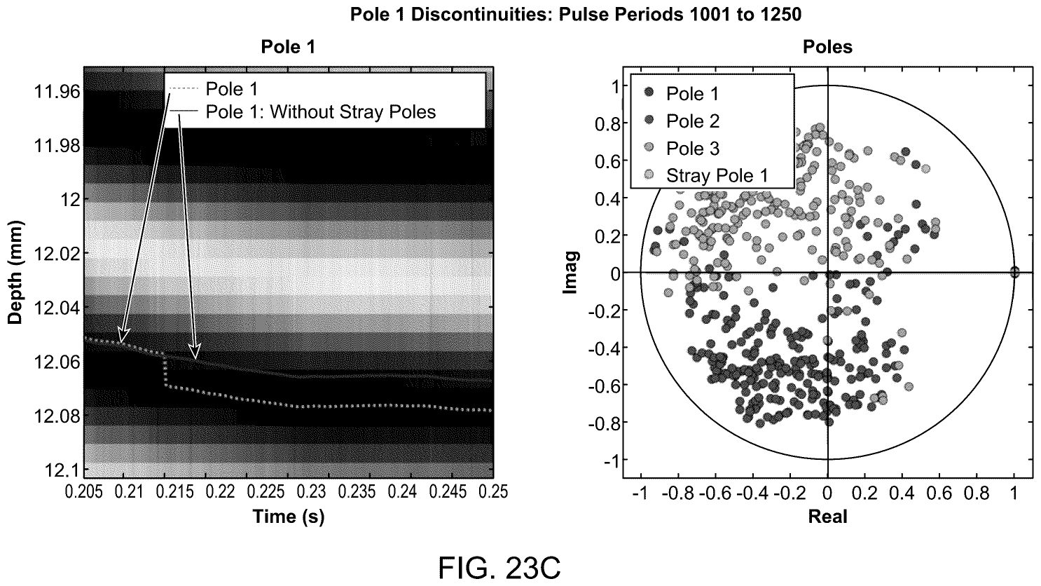

In some cases, it may not be advantageous to pick the first pole (herein “pole number 1”) in autoregression: however, this may not be the pole which returns the smallest absolute error. Disclosed herein are improved methods for pole selection in autoregression. For example, FIG. 17 demonstrates exemplary velocity and position traces that result from always selecting pole 1 of the AR model. In some cases, there can be discontinuities that result in the velocity and position traces (FIG. 23A-C).

FIG. 23A, FIG. 23 B, and FIG. 23C show examples of regions where the pole 1 trace shows a discontinuity over three different ranges of pulse periods, 251-500, 751-1000, and 1001-1250, respectively.

FIG. 23A (left) shows an exemplary pole 1 position trace and pole 1 position trace with discontinuous poles removed plotted on the RF phase tracks. FIG. 23A (right) shows exemplary autoregression poles 1, 2, and 3 plotted on the complex unit circle. When pole 1 is discontinuous, for example, a different pole can be selected.

The decision to select pole 1, pole 2, or pole 3 may be made using one or more of the discriminators listed below in Table 2 and Table 3.

TABLE 2
Discriminators for AR pole selection algorithms

| Discriminator | Mathematical Formulation | Description |
|---|---|---|
| Pole Radius | Given Pole: z = a + bi | The distance from (0, 0) to a |
| | radius(z) = \sqrt{a² + b²} | given pole on the complex unit |
| | | circle. |
| Pole Angle | Given Pole: z = a + bi | The angle formed between |
| | Given a 4 quadrant arctan function | (1, 0), (0, 0), and a given pole on |
| | $angle ( z ) = arctan ( \frac{b}{a} )$ | the complex unit circle. A pole z = 1 + 0i has an angle |
| | | of 0 radians. |
| | | A pole z = −1/2 + (√3/2)i has an |
| | | angle of 2π/3 radians. |
| | | A pole z = −1/2 − (√3/2)i has an |
| | | angle of − 2π/3 radians. |
| A priori | If ΔPressure > 0 Then sign(Velocity) = +1 | If the sign of the derivative of |
| Pressure | If ΔPressure < 0 Then sign(Velocity) = −1 | the pressure change is positive, |
| Information | | then physically the velocity of |
| | | the TM should also be positive. |
| | | Similarly, if the sign of the |
| | | derivative of the pressure |
| | | change is negative, then the |
| | | velocity of the TM should be |
| | | negative. |
| Complex | Given a Previous Pole: z₁ = a + bi | The distance from one pole to |
| Plane Distance | & | the next on the complex plane |
| | Given a Current Pole: z₂ = c + di | using the Pythagorean distance |
| | CompDist (z₁ to z₂) = | equation (Euclidean distance). |
| | \sqrt{(c − a)² + (d − b)²} | |
| Change in | Given a Previous Pole: z₁ = a + bi | The change magnitude of the |
| Pole Velocity | & | velocity from one pole to the |
| | Given a Current Pole: z₂ = c + di | next. The velocity of a pole is |
| | ΔV(z₁ to z₂) = angle(z₂) − angle(z₁) | proportional to the angle of a |
| | | pole, so the change in velocity |
| | | from one pole to the next is |
| | | proportional to the change in |
| | | angle. |
| Inertial | Given a Previous Pole: z₁ = a + bi | The change in velocity between |
| Velocity | & | two poles. If the velocity |
| | Given a Current Pole: z₂ = c + di | changes directions from one |
| | ΔVᵢₙₑᵣₜᵢₐₗ (z₁ to z₂) = | pole to the next, a penalty is |
| | angle(z₂) − angle(z₁) | applied to overcome the |
| | if angle (z₂) changes direction or sign | “inertia” associated with that |
| | ΔVᵢₙₑᵣₜᵢₐₗ = ΔVᵢₙₑᵣₜᵢₐₗ + \|angle(z₁)\| | direction change. |
| Momentum: | Given a Previous Pole: z₁ = a + bi | Momentum (P) is equal to |
| Multiplication | & | velocity (v) times mass (m). AR |
| and then | Given a Current Pole: z₂ = c + di | poles have analogs for velocity |
| Subtraction | ΔP(z₁ to z₂) = radius(z₂)angle(z₂) − | and mass, with angle and radius |
| (MtS) | radius(z₁)angle(z₁) | being velocity and mass |
| | if angle (z₂) changes direction or sign | respectively. Pole momentum |
| | ΔP = ΔP + \|angle(z₁)\|radius(z₁) | can therefore be described as P = |
| | | (angle)(radius). |
| | | Change in momentum can be |
| | | written as follows: |
| | | ΔP = P₂ − P₁ |
| | | Pole momentum can be written |
| | | similarly: |
| | | ΔP(z₁ to z₂) = |
| | | radius(z₂)angle(z₂) − |
| | | radius(z₁)angle(z₁) |
| Momentum: | Given a Previous Pole: z₁ = a + bi | Change in momentum can also |
| Subtraction | & | be written as follows: |
| and then | Given a Current Pole: z₂ = c + di | ΔP = mΔV |
| Multiplication | Δm = radius(z₂) − radius(z₁) | Pole momentum can be written |
| (StM) | ΔV = angle(z₂) − angle(z₁) | similarly: |
| | ΔP(z₁ to z₂) = ΔmΔV | ΔP(z₁ to z₂) = |
| | if angle (z₂) changes direction or sign | (radius(z₂) − radius(z₁)) |
| | ΔP = ΔP + \|angle (z₁)\|radius(z₁) | * (angle(z₂) − angle(z₁)) |
| n-Segment | Given a Previous Pole: z₁ = a + bi | The complex distance formula |
| Distance | & | between two poles (show |
| | Given a Current Pole: z₂ = c + di | above) can be extended to give |
| | ... | the entire distance along a path |
| | Given an n + 1 Pole: z₍ₙ₊₁₎ = x + yi | of poles from some previous |
| | n − SegDist = CompDist(z₁ to z₂) + ... + | pole to an n + 1 pole. |
| | CompDist(zₙ to z₍ₙ₊₁₎) | This will return the sum of n |
| | | distances between n + 1 poles. |
| n-Segment | Given a Previous Pole: z₁ = a + bi | The change in velocity formula |
| Change in | & | between two poles (show |
| Velocity | Given a Current Pole: z₂ = c + di | above) can be extended to give |
| | ... | the change in velocity along a |
| | Given an n + 1 Pole: z₍ₙ₊₁₎ = x + yi | path of poles from some |
| | | previous pole to an n + 1 pole. |
| | n − SegΔV = \|ΔV(z₁ to z₂)\| + ... + | This will return the sum of n |
| | \|ΔV(zₙ to z₍ₙ₊₁₎)\| | change in velocity values |
| | | calculated using n + 1 poles. |

TABLE 3
Descriptions of the AR pole selection algorithms

| Algorithm | | | | Otherwise | | |
|---|---|---|---|---|---|---|
| Name | Try | Unless | Otherwise | if | Then | Otherwise |
| Pole 1 with | Pick Pₖ₁ | angle(Pₖ₁) > pi/3 Or | Select the pole | | | |
| Limits | | radius(Pₖ₁) < 0.9 | (Pₖ) that | | | |
| | | | minimizes | | | |
| | | | CompDist(Pₖ₋₁, | | | |
| | | | Pₖ) | | | |
| Pole 1 with | Pick Pₖ₁ | angle(Pₖ₁) > pi/3 Or | Select the pole | The velocity | Select the pole | If no poles |
| Limits and | | radius(Pₖ₁) < 0.9 | (Pₖ) that | of the | that agrees | agree, select |
| a priori | | | minimizes | selected Pₖ | with the a priori | the pole (Pₖ) |
| Pressure | | | CompDist(Pₖ₋₁, | violates the | pressure | that minimizes |
| Information | | | Pₖ) | a priori | information | CompDist(Pₖ₋₁, |
| | | | | pressure | | Pₖ) |
| | | | | information | | |
| Min Complex | Select the pole (Pₖ) that | | | | | |
| Distance | minimizes CompDist(Pₖ₋₁, Pₖ) | | | | | |
| Min Complex | Select the pole (Pₖ) that | | | | | |
| Distance | minimizes CompDist(Pₖ₋₁, | | | | | |
| (2 Segment) | Pₖ) + CompDist(Pₖ, Pₖ₊₁) | | | | | |
| Min Change | Select the pole (Pₙ) that | | | | | |
| in Velocity | minimizes ΔV(Pₙ₋₁, Pₙ) | | | | | |
| Min Change | Select the pole (Pₖ) that | | | | | |
| in Velocity | minimizes ΔV(Pₖ₋₁, | | | | | |
| (2 Segment) | Pₖ) + ΔV(Pₖ, Pₖ₊₁) | | | | | |
| Min Change | Select the pole (Pₖ) that | The velocity of the | Select the pole | No poles | Select the pole | |
| in Velocity | minimizes ΔV(Pₖ₋₁, | selected Pₖ violates | that agrees with | agree with | (Pₖ) that | |
| with a priori | Pₖ) + ΔV(Pₖ, Pₖ₊₁) | the a priori pressure | the a priori | the a priori | minimizes | |
| Pressure | | information | pressure | pressure | CompDist(Pₖ₋₁, | |
| Information | | | information | information | Pₖ) | |
| (2 Segment) | | | | | | |
| Min Change | Select the pole (Pₖ) that | | | | | |
| in Momentum | minimizes ΔP(Pₖ₋₁, Pₖ) | | | | | |
| (MtS) | using MtS conventions | | | | | |
| | where: | | | | | |
| | ΔP(Pₖ₋₁, Pₖ) = | | | | | |
| | ΔP(Pₖ₋₁, Pₖ) + | | | | | |
| | 3*radius(Pₖ₋₁)*angle(Pₖ₋₁) | | | | | |
| | for flipped signs | | | | | |
| Min Change | Select the pole (Pₖ) that | | | | | |
| in Momentum | minimizes ΔP(Pₖ₋₁, Pₖ) | | | | | |
| (StM) | using StM conventions | | | | | |
| | where: ΔP(Pₖ₋₁, Pₖ) = | | | | | |
| | ΔP(Pₖ₋₁, Pₖ) + | | | | | |
| | 3*radius(Pₖ₋₁)*angle(Pₖ₋₁) | | | | | |
| | for flipped signs | | | | | |
| Min Change | Select the pole (Pₖ) that | | | | | |
| in Momentum | minimizes ΔP(Pₖ₋₁, Pₖ) | | | | | |
| (MtS and StM) | using MtS and StM | | | | | |
| | conventions. | | | | | |
| | The resulting pole (Pₖ) is | | | | | |
| | the average of the poles | | | | | |
| | (Pₖ) returned from MtS | | | | | |
| | and StM. | | | | | |
| Pole 1 with | Pick Pₖ₁ | Sign(angle(Pₖ₋₁)) ≠ | Select the pole | | | |
| Discontinuity | | Sign(angle(Pₖ₁)) and | (Pₖ) that | | | |
| Checking | | CompDist(Pₖ₋₁, | minimizes | | | |
| | | Pₖ₁) > 0.05 Or | CompDist(Pₖ₋₁, | | | |
| | | Radius(Pₖ₁) − | Pₖ) | | | |
| | | Radius(Pₖ₋₁) > 0.1 | | | | |
| | | And | | | | |
| | | CompDist(Pₖ₋₁, | | | | |
| | | Pₖ₁) > 0.1 | | | | |
| | | ΔVᵢₙₑᵣₜᵢₐₗ > pi/6 | | | | |
| Pole 1 with | Pick Pₖ₁ | | Select the pole | | | |
| Inertial | | | (Pₖ) that | | | |
| Velocity | | | minimizes | | | |
| Check | | | CompDist(Pₖ₋₁, | | | |
| | | | Pₖ) | | | |
| Pole 1 with | Pick Pₖ₁ | ΔP_{MtS} > pi/3 | Select the pole | | | |
| Momentum | | | (Pₖ) that | | | |
| (MtS) Check | | | minimizes | | | |
| | | | CompDist(Pₖ₋₁, | | | |
| | | | Pₖ) | | | |

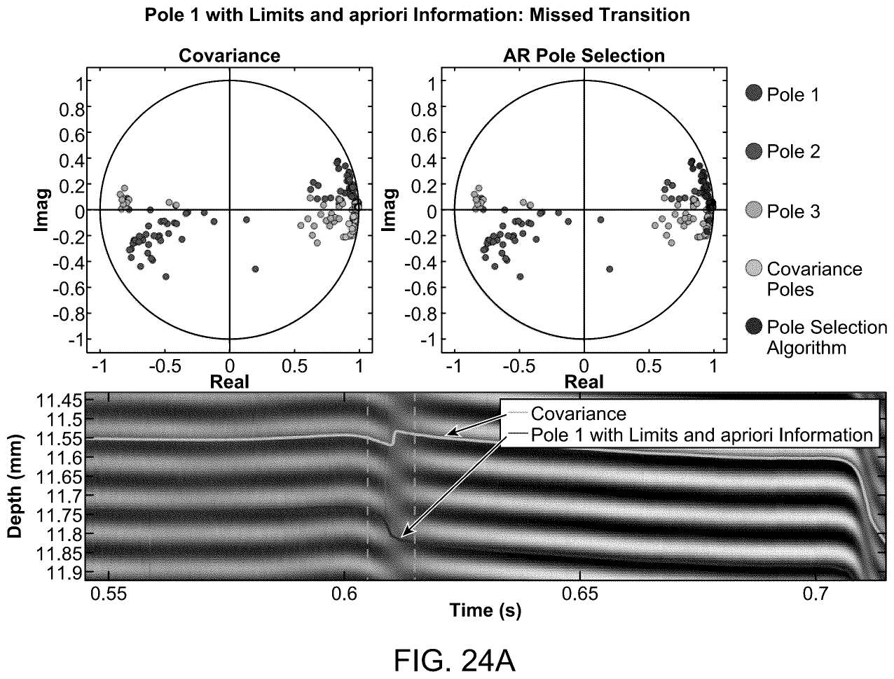

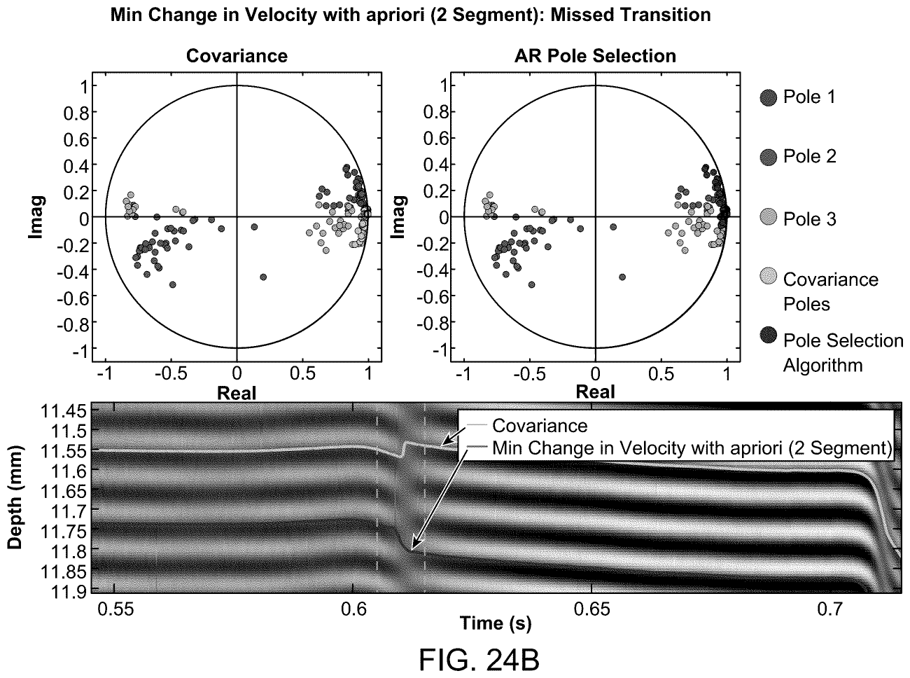

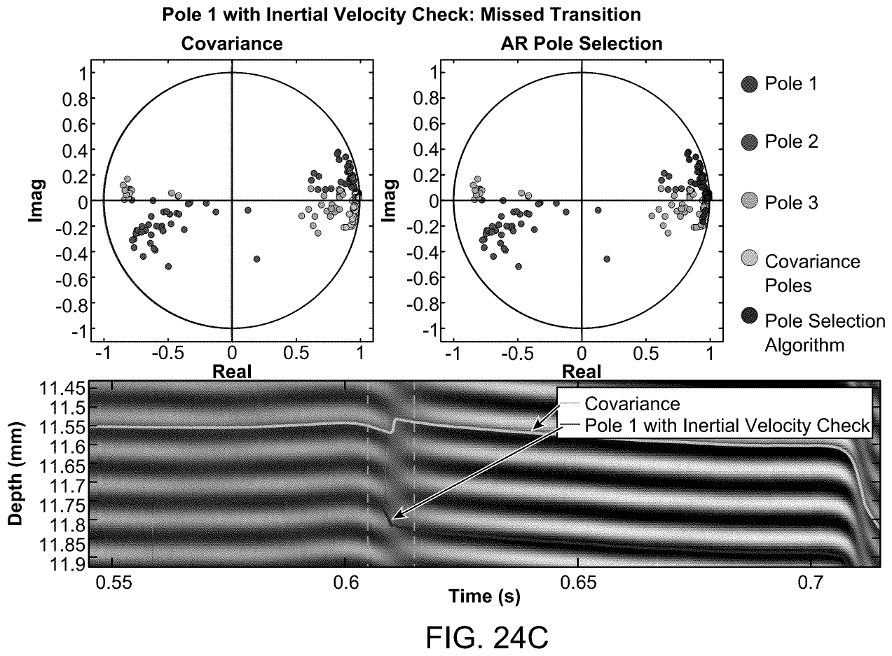

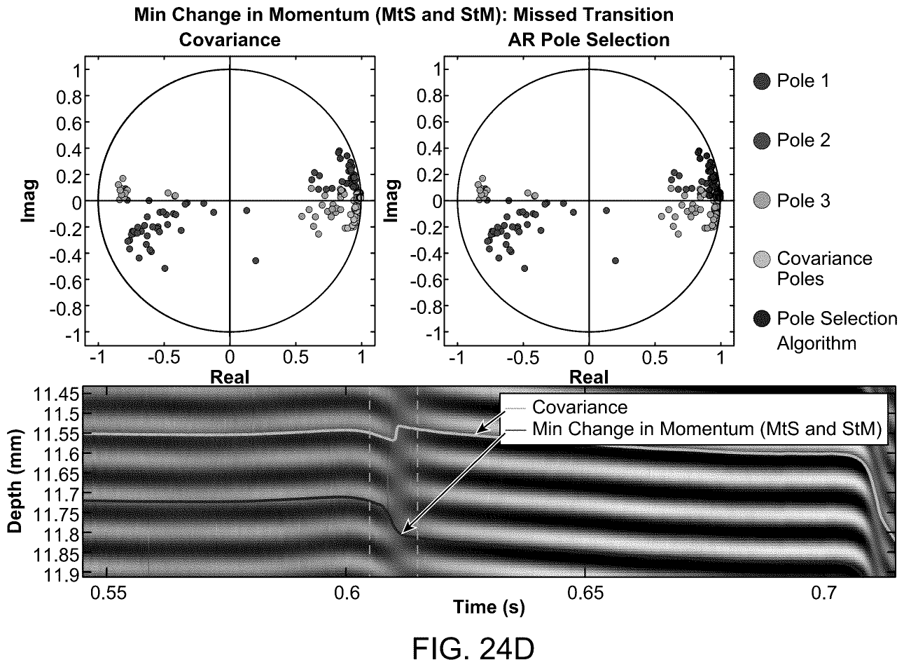

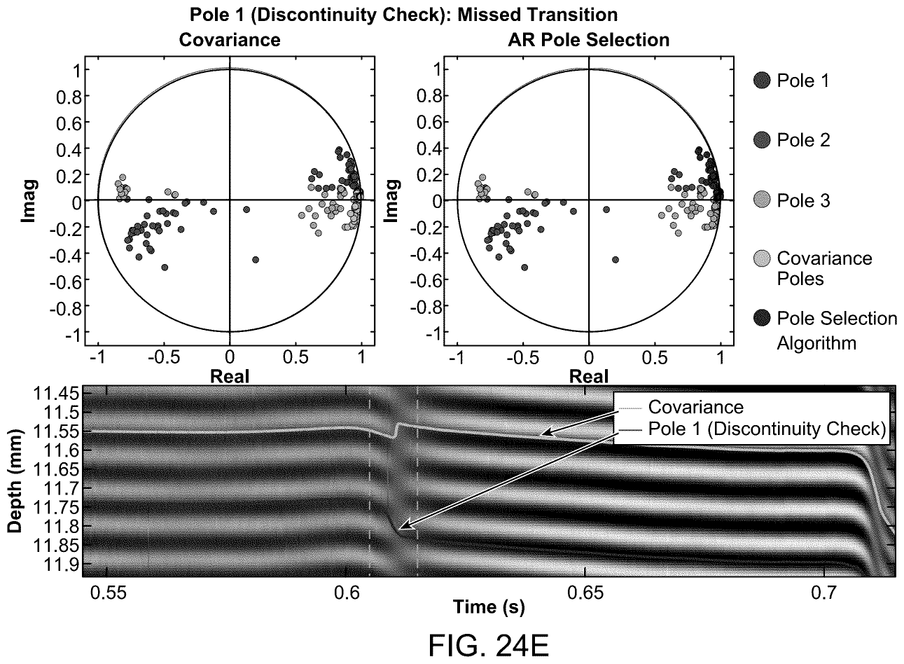

FIG. 24A, FIG. 24B, FIG. 24C, FIG. 24D, and FIG. 24E show five examples of implementation of the AR pole selection algorithms described above.

### Computer Systems

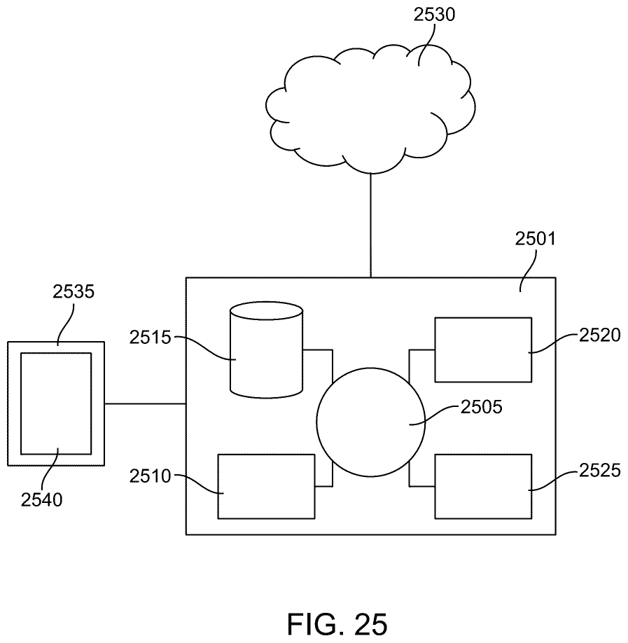

The present disclosure provides computer systems that are programmed to implement methods of the disclosure. FIG. 25 shows a computer system 2501 that is programmed or otherwise configured to perform methods of determining a time-dependent position of a tissue as disclosed herein. The computer system 2501 can regulate various aspects of the otoscopes and systems for determining a time-dependent position of tissue of the present disclosure, such as, for example, transmitting a stimulus, transmitting and/or receiving ultrasound signals, and performing various data analysis operations and sub-operations. The computer system 2501 can be an electronic device of a user or a computer system that is remotely located with respect to the electronic device. The electronic device can be a mobile electronic device.

The computer system 2501 includes a central processing unit (CPU, also “processor” and “computer processor” herein) 2505, which can be a single core or multi core processor, or a plurality of processors for parallel processing. The computer system 2501 also includes memory or memory location 2510 (e.g., random-access memory, read-only memory, flash memory), electronic storage unit 2515 (e.g., hard disk), communication interface 2520) (e.g., network adapter) for communicating with one or more other systems, and peripheral devices 2525, such as cache, other memory, data storage and/or electronic display adapters. The memory 2510, storage unit 2515, interface 2520 and peripheral devices 2525 are in communication with the CPU 2505 through a communication bus (solid lines), such as a motherboard. The storage unit 2515 can be a data storage unit (or data repository) for storing data. The computer system 2501 can be operatively coupled to a computer network (“network”) 2530 with the aid of the communication interface 2520. The network 2530 can be the Internet, an internet and/or extranet, or an intranet and/or extranet that is in communication with the Internet. The network 2530) in some cases is a telecommunication and/or data network. The network 2530 can include one or more computer servers, which can enable distributed computing, such as cloud computing. The network 2530, in some cases with the aid of the computer system 2501, can implement a peer-to-peer network, which may enable devices coupled to the computer system 2501 to behave as a client or a server.

The CPU 2505 can execute a sequence of machine-readable instructions, which can be embodied in a program or software. The instructions may be stored in a memory location, such as the memory 2510. The instructions can be directed to the CPU 2505, which can subsequently program or otherwise configure the CPU 2505 to implement methods of the present disclosure. Examples of operations performed by the CPU 2505 can include fetch, decode, execute, and writeback.

The CPU 2505 can be part of a circuit, such as an integrated circuit. One or more other components of the system 2501 can be included in the circuit. In some cases, the circuit is an application specific integrated circuit (ASIC).

The storage unit 2515 can store files, such as drivers, libraries and saved programs. The storage unit 2515 can store user data, e.g., user preferences and user programs. The computer system 2501 in some cases can include one or more additional data storage units that are external to the computer system 2501, such as located on a remote server that is in communication with the computer system 2501 through an intranet or the Internet.

The computer system 2501 can communicate with one or more remote computer systems through the network 2530. For instance, the computer system 2501 can communicate with a remote computer system of a user (e.g., providing processed data, providing updates, providing use instructions, troubleshooting, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2501 via the network 2530.

Methods as described herein can be implemented by way of machine (e.g., computer processor) executable code stored on an electronic storage location of the computer system 2501, such as, for example, on the memory 2510 or electronic storage unit 2515. The machine executable or machine-readable code can be provided in the form of software. During use, the code can be executed by the processor 2505. In some cases, the code can be retrieved from the storage unit 2515 and stored on the memory 2510 for ready access by the processor 2505. In some situations, the electronic storage unit 2515 can be precluded, and machine-executable instructions are stored on memory 2510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2501 can include or be in communication with an electronic display 2535 that comprises a user interface (UI) 2540 for providing, for example, an indication of a characterization of a state or condition of a tympanic membrane, one or more traces relating to a tissue position or motion, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2505. The algorithm can, for example, calculating from the ultrasound data a covariance matrix from a group of depths and between a plurality pulse periods of the ultrasound data; calculating one or more eigenvalues of the covariance matrix; analyzing a frequency content of the ultrasound data across a plurality of pulse periods; using the frequency content to calculate a second trace of a position of the tissue; determining one or more regions where the first trace comprises a non-physical tissue position or movement; substituting at least a portion of the second trace for the first trace in the one or more regions; etc.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### Methods and Systems Applications

The methods and systems as disclosed herein may be used to characterize a number of biological tissues to provide a variety of diagnostic information. A biological tissue may comprise a patient organ. A speculum may be disposed within a bodily cavity to characterize a patient tissue. A patient organ or bodily cavity may comprise, for example, a muscle, a tendon, a ligament, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, a testicle, a prostate, a heart, an artery, a vein, a spleen, a gland, a brain, a spinal cord, a nerve, etc., to name a few.

The methods and systems as disclosed herein may be used to classify a tympanic membrane. For example, a membrane may be classified to determine a condition of an ear, such as acute otitis media (AOM), chronic otitis media, otitis media with effusion and/or chronic suppurative otitis media. A classification that an ear exhibits AOM may include detection of the presence of effusion and characterization of the type of effusion as one of serous, mucoid, purulent, or combinations of these. In AOM, the middle ear effusion (MEE) may be induced by infective agents and may be thin or serous with viral infection and thicker and purulent with bacterial infection. Accordingly, determining various properties of a fluid adjacent a tympanic membrane may provide information which may be used to characterize a membrane. For example, an ultrasound transducer may be provided on a speculum and positioned within the ear canal. An excitation generator may apply an impulsive pressure to the tympanic membrane, the transducer may direct ultrasound to the tympanic membrane, and reflected ultrasound energy may be measured from the surface of tympanic membrane. The phase changes of the reflected ultrasound during application of the non-contact excitation and/or after removal of the non-contact excitation may indicate an elasticity which may be correlated to the type of fluid behind the tympanic membrane. e.g., air, indicating a healthy ear, clear fluid, indicating a viral infection, or opaque fluid, indicating a bacterial infection.

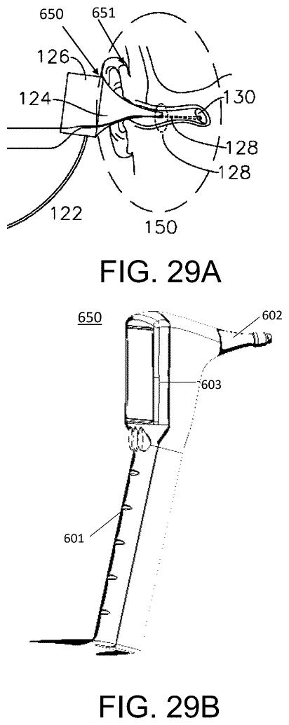

FIG. 29A illustrates a side section view a speculum of an otoscope 650 disposed within an ear 651, in accordance with some embodiments. FIG. 29B illustrates a front section view of an otoscope 650 of the present disclosure, in accordance with some embodiments. Region 150 (shown in magnified view FIG. 29A) illustrates a cross section view of a middle ear and tympanic membrane 130 of a subject being examined. The tympanic membrane 130 may be interrogated by an ultrasound beam 128 from an ultrasound transducer. The transducer may be mounted on the inner surface of a speculum tip 124. The speculum tip may be detachable from an otoscope 650 via speculum mounting adapter 126. The speculum tip may be operably coupled or include an excitation generator. In some cases, the excitation generator may create a pressure excitation. In some cases, the excitation generator may create pressure excitation which is a sonic excitation, a sub-sonic excitation, or a super-sonic excitation. The pressure excitation generated by the excitation generator may be an impulsive step or delta (impulse) generation, a sinusoidal pressure excitation, a square wave excitation, or any combination of these, and the excitation may be a gated burst or continuous. The pressure excitation may be provided with or without a static positive or negative pressure bias.

In some examples, an excitation generator creates a pressure excitation, such as an air puff. For instance, the otoscope mounting adapter 126 and speculum tip 124 may have a common interior volume. The common interior volume may provide for coupling of dynamic pressures from an excitation generator through a coupling 122 to the ear canal where the air pressures result in displacement of the tympanic membrane 130. The excitation generator may generate pressure variations which are coupled into the ear canal through the speculum tip 126.

In some examples, an excitation generator may be an air bladder manipulated by an operator to apply a force to a membrane or surface, an air displacement generator producing alternating pressure, step pressure, or air puffs. The excitation generator output may be sealed to the surrounding region of the surface or unsealed and using a puff of gas such as atmospheric air or other suitable gas.

In some examples, an excitation generator may produce a sonic excitation, a sub-sonic excitation, or a super-sonic excitation. For example, the excitation generator may produce a sub-audio frequency below 20 Hz, an audio frequency from 20 Hz to 20 KHz, or a super-audio frequency above 20 KHz. In an example, a sonic excitation, a sub-sonic excitation, or a super-sonic excitation may be produced by a piezoelectric transducer. The piezoelectric transducer may convert an electrical signal to a physical displacement which may in turn induce a pressure wave. In an example, a sonic excitation, a sub-sonic excitation, or a super-sonic excitation may be produced by a cMUT transducer. In an example, an audio speaker with a voice-coil actuator may be used to produce an excitation.

An ultrasound transducer may be provided within an otoscope in addition to and distinct from the excitation generator. The ultrasound transducer may comprise a variation, example, or embodiment of any transducer element or ultrasound transducer disclosed herein. In some cases, the ultrasound transducer and the excitation generator may be the same element.

As shown in FIG. 29B, an otoscope may comprise a handle 601 for positioning of the speculum 602. The otoscope may include a video display 603. The display may display to a user an optical image of the membrane to be characterized. The display 603 may show an ultrasound image. The display 603 may provide a user interface in which to control various aspects of the otoscope 650) and/or analysis of the ultrasound data. The otoscope may comprise a digital processing device on board, for example, within a handle 601 of the device. The otoscope may connect to a remote device such as a server, a remote memory, a remote processing device, etc. The analysis of the ultrasound data may be performed on board or remotely.

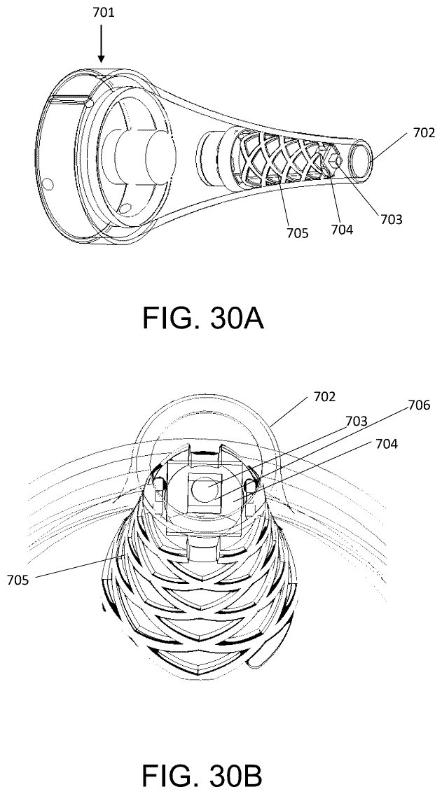

FIG. 30A illustrates a side section view of a speculum 701, in accordance with some embodiments. FIG. 30B illustrates a front section view of a tip of a speculum 702, in accordance with some embodiments. In some examples, the ultrasound transducer 703 is disposed within the speculum 701. The speculum may be disposable. The speculum may comprise an ultrasound transducer 703 disposed near a tip region 702 of the speculum 701. The speculum may comprise a lens assembly 704 which may aid in providing optical images to a user to guide positioning of an ultrasound transducer. In some examples, the ultrasound transducer 703 may be at the center of the speculum, and the optical sensing is consequently accomplished around the ultrasound transducer 704. The ultrasound transducer may be supported by a mesh 705. The mesh 705 may allow transmission of electrical signals to a digital processing device.

Ultrasound transducers described herein may comprise a base 706. The base may be mounted within the speculum via a plate 704. The plate 704 may allow the transducer to be centered or approximately centered within the opening of the otoscope. The plate 704 may be optically transparent. In an example, the plate 704 is glass. The plate 704 may comprise one or more openings which may allow for a pressure excitation to be conveyed from the interior of the speculum tip to the exterior of the speculum tip. The plate 704 may comprise one or more electrically conductive portions to allow for a driving voltage and/or current to be supplied to the transducer. The plate 704 may comprise one or more insulating layers. The plate 704 may itself comprise conducting or insulating portions. The plate 704 may be insulating with conducting portions mounted thereon.

In another example, the methods and systems as disclosed herein may be used to characterize an animal or human organ such as the eye. For example, the excitation generator may apply an impulsive pressure to an eye, the transducer may direct ultrasound to the eye, and reflected ultrasound energy may be measured from the surface of the eye. The phase changes of the reflected ultrasound during application of the non-contact excitation and/or after removal of the non-contact excitation may indicate an elasticity which may be correlated to an inter-ocular pressure for measurement or diagnosis of glaucoma.

In another example, the methods and systems as disclosed herein may be used to characterize an animal or human lung. For example, audio tones from the chest (for example, at a frequency of 3-20 Hz) could be demodulated from the transducer. The transducer could be integrated into a stethoscope-like device wherein the transducer can be moved up the chest during a “knock test” (auscultation) to identify a change in the reflected ultrasound which can indicate fluid in the lungs (e.g., mucus or water). In some cases, a plurality or array of transducers may be provided and placed or worn on the chest. The phase changes of the reflected ultrasound during application may indicate a change in fluid viscosity which may be correlated to a lung disease such as pneumonia, lung cancer, chronic obstructive pulmonary disease (COPD), idiopathic pulmonary fibrosis (IPF), etc.

The methods and systems as disclosed herein may be used for example, to characterize a food item. For example, the excitation generator may apply an impulsive pressure to the surface of a food item such as a vegetable or fruit, and the ultrasound energy may be applied to the food item to measure the time dependent surface response of the fruit or vegetable, to determine an elasticity or other physical property which may be correlated to the ripeness of the fruit or vegetable. For example, the food item may be placed into a holder and the surface excited with a puff of gas such as air, the surface deflection response estimating ripeness or other property. For example, the excitation may be a gas which may be delivered at a supersonic velocity and/or at a glancing angle to the surface of the food item, or one or more food items may be placed into a chamber which has a variable pressure to measure a low frequency surface response to pressure, such as deflection vs. pressure. For example, the excitation may be applied to one surface and the response measured on a different surface of the same item, such as the measurement of a propagating surface wave or a shear wave which travels through the item being characterized.

The methods and systems as disclosed herein may be used to characterize an industrial process. For example, the small size of a transducer disclosed herein can be applied to any industrial process wherein larger transducers, ultrasound, or other modalities such as LIDAR are prohibitive due to their large size. The high resolution achieved by the presently disclosed transducer at short ranges, e.g., less than 25-35 millimeter range, with 10-20 micrometer movement (e.g., via Doppler integration) allows the present invention to be applied to a wide array of industrial processes wherein analysis without physically touching the analyte is necessary. For example, the excitation generator may apply an impulsive pressure to the surface of a manufactured part such as to determine the consistency of a viscous fluid such as a lubricant, and the ultrasound energy may be applied to the part to measure the time dependent surface response of the viscous fluid, to determine an elasticity or other physical property which may be correlated to quality of the lubricant. The transducer may be used to measure the thickness of paint by comparing a painted section of an object to an unpainted section. The transducer may be used to measure whether a painted object is dry by comparing a painted object to a similar object that has been recently painted with an identical paint. The transducer may be used as a part of a manufacturing process to identify an object as a part of counting the objects being manufactured. The transducer may be used to measure a change in density or composition of an object by comparing an object that has undergone a process to an object before the process (e.g., cooked food items, curing processes, etc.). Other industrial examples may include range finding applications, ultrasonic transit-time gas flow meters for metering dynamic gas flows, anemometry applications, and various other ultrasound-based sensing applications.

The methods and systems as disclosed herein may comprise using an ultrasound transducer; directing the tip of a speculum within a lumen adjacent the membrane; directing a perturbation to a surface of the membrane; measuring a reflected ultrasound signal from the surface of the membrane; and characterizing the viscosity or elasticity of the membrane in response to the perturbation and the reflected ultrasound.

### EXAMPLES

### Example 1: Analysis of Covariance Matrices Across Phase Tracks

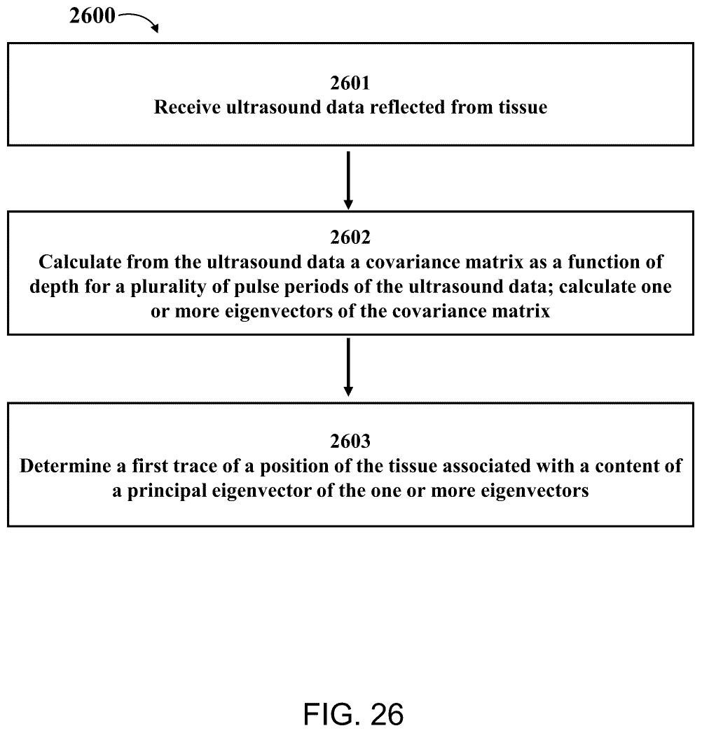

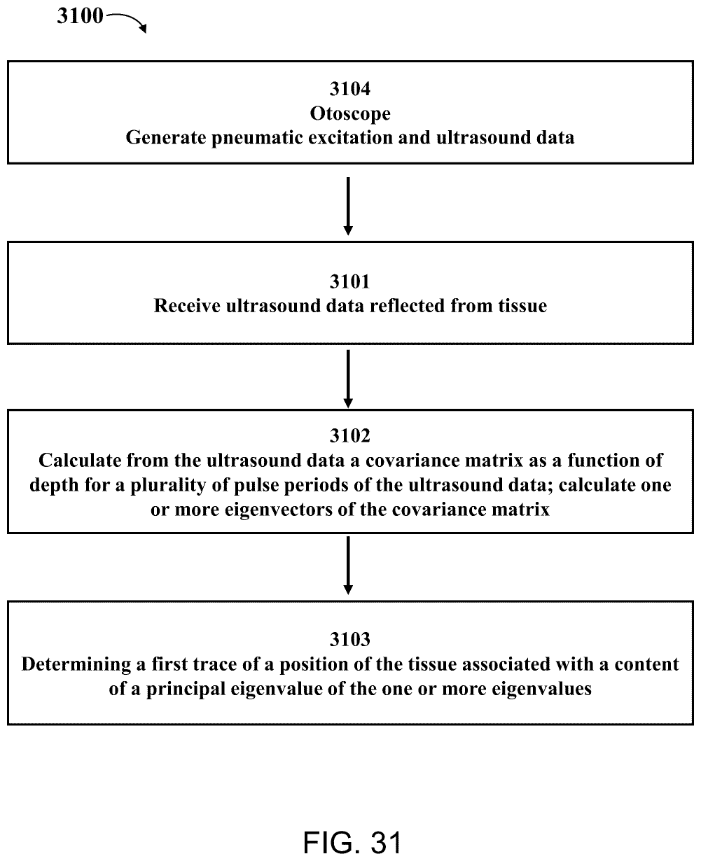

In some non-limiting examples, covariance matrixes were used to determine a time-dependent position of a tissue in response to a stimulus 2600 (FIG. 26). In some non-limiting examples, covariance matrixes were used to determine a time-dependent position of a tissue 3100 in response to a pneumatic excitation generated by an otoscope 3104 (FIG. 31).

Ultrasound data, derived from an ultrasound waveform reflected from a tissue, was received for analysis 2601. From the ultrasound data, a covariance matrix was calculated from a group of depths and for multiple pulse periods of the ultrasound data, and from the covariance matrix one or more eigenvectors was calculated 2602. The covariance matrix was constructed by taking the Hilbert transformed RF ultrasound samples within a clear echo region of ultrasound data. The covariance matrix was analyzed by a Hilbert transform; for example, the analytic pulse echo signal that results from the Hilbert transform of a single pulse period, denoted by “k” for kth pulse period, is expressed as:

$$H ( z , k ) = H ( m , k ) = R ( m , k ) exp ( j [ ω t + φ ( m , k ) ] )$$

where R is amplitude, ω=2πf is the carrier frequency, φ is signal phase, and z is depth and corresponds to the m^{th} a/d sample. The analytic pulse echo signal was used to find elements of the covariance matrices. The covariance matrices were from the same depth but two different pulse periods. These pulse periods are each a known number of pulse periods from pulse period k, and are denoted by indices p and q. The covariance matrix summand arose from a fixed depth, z or m. The covariance matrix can be an outer product matrix described by

$$K ( p , q , m ) = H ( m , p ) * {H}^{′} ( m , q )$$

where p={0 . . . N−1} and q={0 . . . N−1}, and the apostrophe denotes complex conjugation.

The completed covariance matrix sums K(p, q, m) from a group of adjacent depths m={m0, m1, m2, . . . , mR}. Plugging in H from above into this covariance expression yielded the following:

$$K ( p , q , m ) = R ( m , p ) exp ( j [ ω t + φ ( m , p ) ] \cdot R ( m , q ) exp ( - j [ ω t + φ ( m , q ) ] )$$

which is further simplified to:

$$K ( p , q , m ) = R ( m , p ) R ( m , q ) \cdot exp ( j [ φ ( m , p ) - φ ( m , q ) ] ) .$$

The covariance matrix arising from contributions across a series of contiguous depths spanning the specific tympanic membrane echo is denoted by:

$$K ( p , q ) = {\sum }_{m = m 0}^{mR} R ( p , m ) \cdot R ( q , m ) \cdot exp ( j [ φ ( m , p ) - φ ( m , q ) ] )$$

which is written more simply as

$$K ( p , q ) = {\sum }_{m = m 0}^{mR} {R}_{m} ( p ) {R}_{m} ( q ) {e}^{j Δ}$$

where

$$Δ = Δ ( p , q ) = {φ}_{m} ( p ) - {φ}_{m} ( q ) .$$

and Δ is a difference in phase sampled at one depth, m, at two different times, p and q. This is also known as the time rate of change in phase of an ultrasound signal. This can avoid demodulation because the carrier frequency at the start of the description of K is effectively subtracted out by multiplication of one exponential by a second which has been conjugated.

The covariance matrix was calculated based on the above mathematical description. With the resulting matrix, the eigenvectors and eigenvalues are denoted by:

$$K \vert {v}_{1} , {v}_{2} , {v}_{3} … {v}_{N} \vert = \vert \begin{matrix}{λ}_{1} & 0 & & 0 \\ 0 & {λ}_{2} & ⋱ & 0 \\ & 0 & 0 & {λ}_{N}\end{matrix} \vert \vert {v}_{1} , {v}_{2} , {v}_{3} … {v}_{N} \vert .$$

where v₁, v₂, v₃ . . . v_{N} is the set of eigenvectors and (λ₁, λ₂, λ₃ . . . ) are the eigenvalues

The overall signal energy was clustered in just a small number of eigenvectors. The eigenvectors {v₁, v₂, v₃ . . . v_{N}} constitute an orthonormal basis set: the ratio of each eigenvalue to the sum of all eigenvalues is the fraction of signal class energy represented in the associated eigenvector. We found from observing eigenvalue behavior in the case of signals from the tympanic membrane, that the eigenvalue energy is generally (>99%) contained within one eigenvector, and that places where the signal is energy depleted will show a concurrent drop in the energy fraction contained in the first eigenvector. Specifically, a target signal quality ratio is expressed by

$$M = {λ}_{1} / {\sum }_{m = 1}^{N} {λ}_{m}$$

M was evaluated at various depths along the ultrasound beam over the number of pulse periods contained in the covariance calculation. When there was no target present, M could be a value between 0 and 1. In some cases, M could have higher variance than when a target is present.

The behavior of noise process covariance (no target present) resulted in many eigenvectors of covariance needed to describe the variety of motion seen across the sample volume, and this contrasts with the behavior of the target covariance (target present), which resulted in 1 or very few eigenvectors needed to describe the narrow variety of motion seen across the sample volume. The principal eigenvector (largest λ) contained a description of target motion which had contribution from all the a/d samples that were within the target echo. The target motion information was embedded in the covariance principal eigenvector. Analysis of the eigenvectors included repeating the analysis in 10 millisecond (ms) steps in an adjustable 20 ms analysis window. Analyzing the eigenvectors also included analyzing depth range values and creating a set of principal eigenvectors at each 10 millisecond (ms) step.

The set of principal eigenvectors that resulted, 1 per window position, at each of 100 windows in 10 ms increments across the 1 second time range, is represented as

$$V = \{ {v}_{1}^{( 1 )} , {v}_{1}^{( 2 )} , {v}_{1}^{( 3 )} , … , {v}_{1}^{( N )} \} .$$

The membrane motion information contained in an eigenvector was obtained by the following expression

$${\vec{d}}_{k} = \frac{U ( ∠ {v}_{1}^{( k )} ) λ}{4 π}$$

where U is the “phase unwrap operator”. This expression indicated the unwrapped eigenvector phase, factored by one wavelength of carrier frequency for every 4π radians of phase change. \vec{d} is a vector which has one value for every pulse period included in the covariance analysis window.

Similar to the set of eigenvectors above, there was a set of displacement vectors which can be denoted by

$$D = \{ {\vec{d}}_{1} , {\vec{d}}_{2} , {\vec{d}}_{3} , … , {\vec{d}}_{N} \} .$$

The set D of vectors containing positional change during each covariance calculation time interval showed the relative position change made by the tympanic membrane and were “stitched” to their adjacent neighbors, which indicated the tympanic motion. A first trace of a position of the tissue associated with the content or the one or more eigenvalues was then determined 2603.

### Example 2: Deconstruction of Signals Using Autoregression

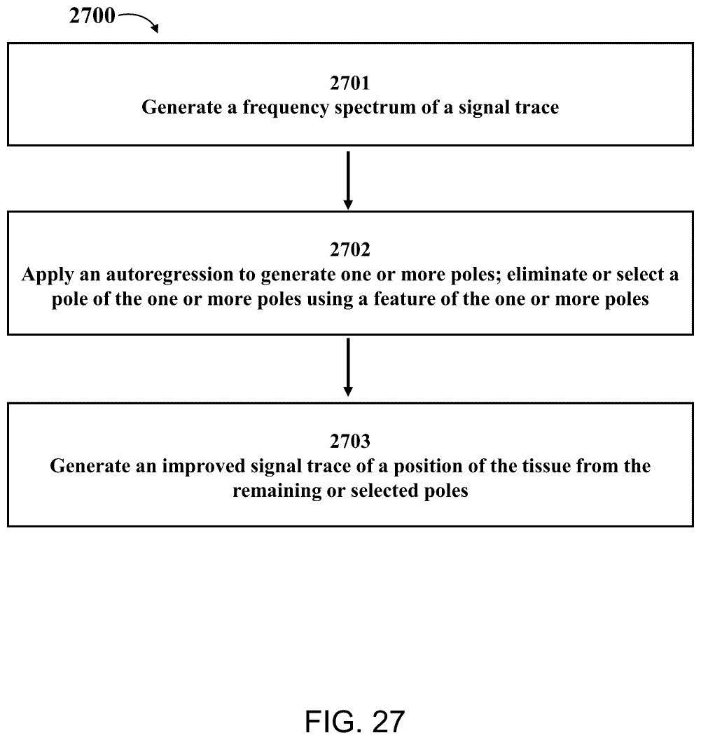

In some non-limiting examples, autoregression (AR) models were used to estimate the frequency spectrum of a signal trace 2701. The autoregression was applied to the eigenvectors of covariance analysis to estimate the frequency spectrum of the signal trace (FIG. 27). The autoregression was, for example, a third order autoregression or, in some non-limiting examples, a higher than third order autoregression. One or more autoregression models were applied to generate one or more poles, and one or more poles were eliminated or selected using one or more features of the one or more poles, for example calculated frequency characteristics 2702. The pulse-repetition frequency was, for example, 5 kHz. The shift frequencies of the ultrasound data was, for example, +/−2.5 kHz. The angles of the complex unit circles representing the ultrasound shift frequencies were, for example, +/−π. The angles also represented velocities of, for example, +/−227.9 mm/s of sound in air. The velocity estimate was calculated by multiplying the radian angle of each pole on the complex unit circle by 72.54 (mm/s)/radian. The radius of each pole was used to determine the variance of the estimated frequency spectrum.

The variance of the estimated frequency spectrum was calculated using the equation:

$$f = \frac{1}{2 π T} {tan}^{- 1} ( \frac{Im ( Pole )}{Re ( Pole )} ) v = 72.54 ( \frac{\frac{mm}{s}}{radian} ) * angle ( Pole )$$

$${σ}^{2} = \frac{2}{{T}^{2}} ( 1 - r )$$

where T is the pulse period, and r is the pole radius.

The third order autoregression model was applied to the first eigenvector of a covariance of Example 1. Pole 1 was determined to primarily estimate the lower frequency velocity of the ultrasound data, and poles 2 and 3 were found to primarily estimate velocities from high frequency ultrasonic data.

The third order autoregression poles were found by solving for the roots of the denominator of a third order estimate for the power spectral density using the power spectral density equation:

$${P}_{3} ( z ) = \frac{{σ}_{noise}^{2}}{{\vert 1 - a ( 1 ) {z}^{- 1} - a ( 2 ) {z}^{- 2} - a ( 3 ) {z}^{- 3} \vert}^{2}}$$

The zeroes of the third order polynomial in the denominator of the power spectral density equation using the poles of the third order equation:

$$A = \frac{\sqrt[3]{- 2 {p}^{3} + 9 pq - 27 r + 3 \sqrt{3} \sqrt{- {p}^{2} {q}^{2} + 4 {q}^{3} + 4 {p}^{3} r - 18 pqr + 27 {r}^{2}}}}{3 \sqrt[3]{2}}$$

$$B = \frac{- {p}^{2} + 3 q}{9 A}$$

$$Root 1 : {a}_{1} = - \frac{p}{3} + A - B$$

$$Root 2 : {a}_{2} = - \frac{p}{3} + ( \frac{- 1 - i \sqrt{3}}{2} ) A - B ( \frac{- 1 + i \sqrt{3}}{2} )$$

$$Root 3 : {a}_{3} = - \frac{p}{3} + ( \frac{- 1 + i \sqrt{3}}{2} ) A - B ( \frac{- 1 - i \sqrt{3}}{2} )$$

The poles of the third order equation were used to determine the frequency estimation of the ultrasonic data, and the poles of the third order autoregression.

The poles of the third order equation in the autoregression model was applied to the first eigenvector of the covariance result to yield three poles. Pole 1 is, for example, a frequency estimator with a lowpass filtering character. Poles 2 and 3 are, for example, frequency estimators with high pass filtering character.

Velocity over time was determined by plotting the angle of a given pole for all pulse periods. The pole angle was calculated by finding the inverse tangent over the imaginary component of a given pole over the real component of a given pole. The poles of the third order equation and the power spectral density equation were applied to convert the angle of a given pole to velocity. The velocities estimated by pole 1, for example, were integrated over time to find displacement. The pole 1 position trace was compared to the covariance position trace to find displacement magnitude. The selection or elimination of the one or more poles was used to generate an optimized signal trace of a position of the tissue from the remaining or selected poles 2703.

### Example 3: Selecting AR Poles

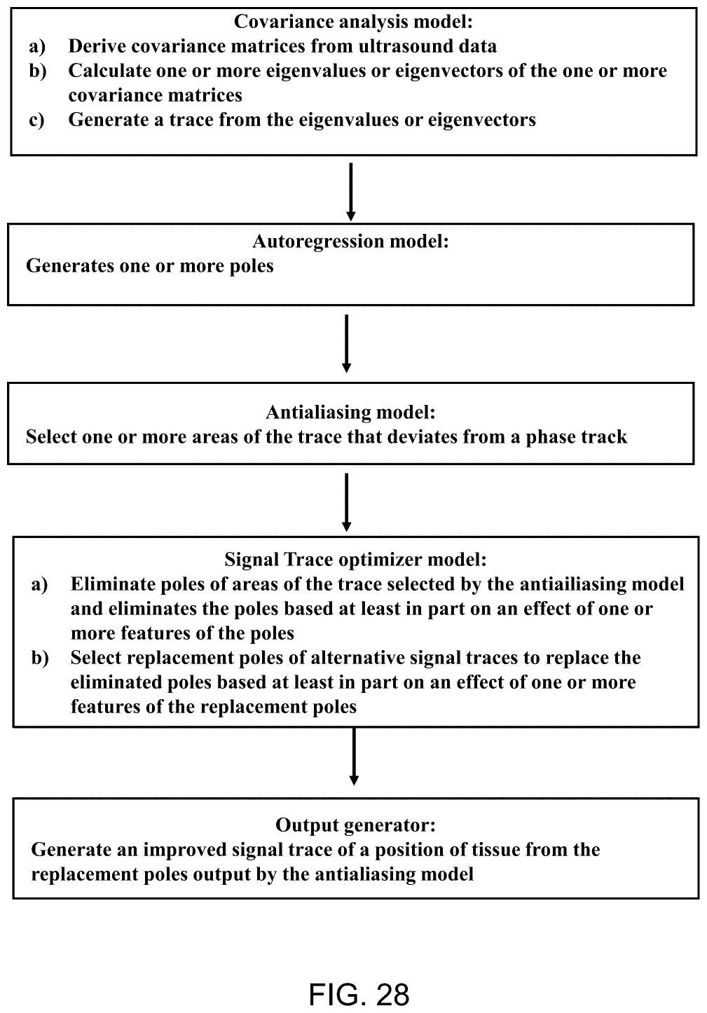

In one non-limiting example, a pole, for example pole 1, was found to be discontinuous, and a different pole was selected to improve the trace (FIG. 28). Multiple autoregression algorithms were applied to select the pole (FIG. 28). The ultrasound measurements were, for example, 5000 pulse periods long. The pulse-repetition frequency was, for example, 5 KHz. The total measurement time was one second. The 12 point autoregression analysis was applied across the first 4800 pulse periods, and the data of the last 200 pulse periods was not included. The first pulse period, for example, pole 1 was automatically selected. The remaining pulse periods, for example, had pole 1, pole 2, pole 3, as choices for selected poles. One or more discriminators as listed in Table 2 and Table 3 were applied to each of the poles at each of the 4799 pulse periods after the first pulse period. The predictive antialiasing model was applied to select areas of the trace that do not follow a desired phase track (FIG. 28).

### Example 4: Antialiasing Methods Operating on Covariance and Eigenvector Motion Detection

In some non-limiting examples, autoregression was applied to an area of a trace where the covariance missed a pressure transition to create an antialiasing fixup trace (FIG. 28). The difference between the absolute errors of the covariance velocity traces and the autoregression traces were determined. The areas where the difference between the absolute errors was greater than π/3, and the velocities were opposite signs, the covariance displacement trace was replaced with the autoregression trace (FIG. 28). An optimized signal trace of a position of tissue from the poles from the antialiasing model was output (FIG. 28).

The absolute values of the error between the autoregression velocities and covariance velocities were measured and logged in an antialiasing fixup chart (FIG. 21).

What is claimed is:

1. A system for determining a time-dependent position of a tissue in response to a stimulus, the system comprising a processor comprising executable instructions stored thereon which when executed are configured to:
(a) receive ultrasound data, wherein the ultrasound data is derived from an ultrasound waveform reflected from the tissue;
(b) calculate from the ultrasound data a covariance matrix from a group of depths and for a plurality of pulse periods of the ultrasound data;
(c) calculate one or more eigenvectors and associated eigenvalues of the covariance matrix; and
(d) determine a first trace of a position of the tissue associated with a content of a principal eigenvector of the one or more eigenvectors.

2. The system of claim 1, wherein the processor is further configured to:
(ii) analyze a frequency content of the ultrasound data across the plurality of pulse periods; and
(ii) calculate a second trace of a position of the tissue based at least in part on the frequency content.

3. The system of claim 1, wherein the processor is further configured to output an indication of a disease state, a health state, or an undetermined state of the tissue in response to the second trace of the position of the tissue.

4. The system of claim 1, wherein the system further comprises a pneumatic otoscope.

5. The system of claim 4, wherein the processor is operatively connected to the pneumatic otoscope.

6. The system of claim 1, further comprising a capacitive micromachined ultrasound transducer.

7. The system of claim 6, wherein the processor is operatively connected to the capacitive micromachined ultrasound transducer.

8. The system of claim 6, wherein the capacitive micromachined ultrasound transducer is disposed within an otoscope.

9. The system of claim 1, wherein the plurality of pulse periods is sequential.

10. The system of claim 1, wherein the tissue is a tympanic membrane.

11. The system of claim 1, wherein the processor is further configured to output an indication of a disease state, a health state, or an undetermined state of the tissue in response to the first trace of the position of the tissue.

12. The system of claim 1, wherein each pulse period of the plurality of pulse periods is associated with a related covariance matrix from a plurality of related covariance matrices, and wherein the system further comprises calculating a set of displacement vectors for the plurality of related covariance matrices.

13. The system of claim 12, wherein the plurality of related covariance matrices is associated with pulse periods across contiguous adjacent depths of the ultrasound data.

14. The system of claim 13, wherein the set of displacement vectors for the plurality of related covariance matrices are calculated based on a target quality signal ratio.

15. The system of claim 1, wherein the processor is further configured to:
(i) analyze one or more eigenvectors of the covariance matrix, wherein the one or more eigenvectors each comprise an orbital rotation associated with a phase of the ultrasound data;
(ii) apply a bias to each of the one or more eigenvectors such that the eigenvector circles an origin in phase space; and
(iii) calculate a second trace of the position of the tissues using a resulting biased eigenvector.

16. The system of claim 15, wherein the processor is further configured to analyze the one or more eigenvectors by analyzing membrane motion information of the one or more eigenvectors.

17. The system of claim 16, wherein the processor is further configured to repeat the analysis in 10 ms steps in an adjustable 20 ms analysis window.

18. The system of claim 1, wherein the stimulus is a pneumatic excitation.

19. The system of claim 1, wherein the processor is further configured to:
determine one or more regions where the first trace comprises a non-physical tissue position or movement; and
substitute at least a portion of the second trace for the first trace in the one or more regions.

20. The system of claim 1, wherein the processor is further configured to analyze the frequency content of the complex demodulation of the ultrasound data by applying autoregression.
